SEQUENCE LISTING (67 sequences)

SEQ ID NO: 1 - protein, 7 aa, synthetic construct
PKKKRKV

SEQ ID NO: 2 - protein, 16 aa, synthetic construct
KRPAATKKAGQAKKKK

SEQ ID NO: 3 - protein, 9 aa, synthetic construct
PAAKRVKLD

SEQ ID NO: 4 - protein, 1627 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGSKRPAATKKAGQAKKKKLE

SEQ ID NO: 5 - protein, 1627 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCWMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGSKRPAATKKAGQAKKKKLE

SEQ ID NO: 6 - protein, 1875 aa, synthetic construct
MKRTADGSEFESPKKKRKVSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKVGSGSKRPAATKKAGQAKKKKLE

SEQ ID NO: 7 - protein, 1627 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFINRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGSKRPAATKKAGQAKKKKLE

SEQ ID NO: 8 - DNA, 4884 bp, synthetic construct
  source 1..4884: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctttggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccctaagcaatacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggcagcggaagcaaaaggccggcggccacgaaaaaggccggccaggcaaaaaagaaaaagctcgagtaa

SEQ ID NO: 9 - DNA, 5628 bp, synthetic construct
  source 1..5628: mol_type = other DNA
atgaaacggacagccgacggaagcgagttcgagtcaccaaagaagaagcggaaagtcagttcaaagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccaaatgtgacactgtttatctacatcgccaggctgtatcacctggcaaacccaaggaataggcagggcctgcgcgatctgatcagctccggcgtgaccatccagatcatgacagagcaggagtccggctactgctggcacaacttcgtgaattattctcctagcaacgagtcccactggcctaggtacccacacctgtgggtgcgcctgtacgtgctggagctgtattgcatcatcctgggcctgcccccttgtctgaatatcctgcggagaaagcagagccagctgacctcctttacaatcgccctgcagtcttgtcactatcagaggctgccaccccacatcctgtgggccacaggcctgaagtctggcggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaggctgggagcccctagagccttcaagtactttgacaccaccatcgaccccaagcagtacaggagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtcggcagcggaagcaaaaggccggcggccacgaaaaaggccggccaggcaaaaaagaaaaagctcgagtaa

SEQ ID NO: 10 - DNA, 4881 bp, synthetic construct
  source 1..4881: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaacagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctttggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccctaagcaatacagaagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggcagcggaagcaaaaggccggcggccacgaaaaaggccggccaggcaaaaaagaaaaagctcgag

SEQ ID NO: 11 - DNA, 20 bp, synthetic construct
  source 1..20: mol_type = other DNA
tttcctgcaagaatatctac

SEQ ID NO: 12 - DNA, 20 bp, synthetic construct
  source 1..20: mol_type = other DNA
ttttcctgcaagaatatcta

SEQ ID NO: 13 - DNA, 20 bp, synthetic construct
  source 1..20: mol_type = other DNA
attttcctgcaagaatatct

SEQ ID NO: 14-51 - skipped (38 entries)

SEQ ID NO: 52 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFFCKNIYRPDKFLQCVKNPEDSSCTSEI

SEQ ID NO: 53-56 - skipped (4 entries)

SEQ ID NO: 57 - protein, 5 aa, synthetic construct
SFAMS

SEQ ID NO: 58 - protein, 16 aa, synthetic construct
AISGSGGGTYYADSVK

SEQ ID NO: 59 - protein, 13 aa, synthetic construct
DKILWFGEPVFDY

SEQ ID NO: 60 - protein, 12 aa, synthetic construct
RASQSVSSYLAW

SEQ ID NO: 61 - protein, 7 aa, synthetic construct
DASNRAT

SEQ ID NO: 62 - protein, 10 aa, synthetic construct
QQRSNWPPTF

SEQ ID NO: 63 - DNA, 1491 bp, synthetic construct
  source 1..1491: mol_type = other DNA
atgctcttgttggtgacgagtctcctgctgtgtgaactgccgcacccagcatttcttttgattccggaggtgcagctgttggagtctgggggaggcttggtacagcctggggggtccctgagactctcatgtgcagtctctggattcacctttaacagctttgccatgagctgggtccgccaggctccagggaaggggctggagtgggtctcagctattagtggtagtggtggtggcacatactacgcagactccgtgaagggccggttcaccatctccagagacaattccaagaacacgctgtatctgcaaatgaacagcctgagagccgaggacacggccgtatatttctgtgcgaaagataagattctctggttcggggagcccgtctttgactactggggccagggaaccctggtcaccgtctcctcagcctccggaggcggaggctccggtgggggaggatctgggggaggcggaagcgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaaaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatgctagcatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgaacggaggcggggcaagggccacgacggcctgtatcagggcctgtccaccgccaccaaggatacctacgacgccctgcacatgcaggccctgcccccaaggtga

SEQ ID NO: 64 - protein, 496 aa, synthetic construct
MLLLVTSLLLCELPHPAFLLIPEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKTTTPAPRPPTPAPTIASQPLSLRPEACRPAAGGAVHTRGLDFACDASMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 1392 bp, synthetic construct
  source 1..1392: mol_type = other DNA
atgctcttgttggtgacgagtctcctgctgtgtgaactgccgcacccagcatttcttttgattccggaggtgcagctgttggagtctgggggaggcttggtacagcctggggggtccctgagactctcatgtgcagtctctggattcacctttaacagctttgccatgagctgggtccgccaggctccagggaaggggctggagtgggtctcagctattagtggtagtggtggtggcacatactacgcagactccgtgaagggccggttcaccatctccagagacaattccaagaacacgctgtatctgcaaatgaacagcctgagagccgaggacacggccgtatatttctgtgcgaaagataagattctctggttcggggagcccgtctttgactactggggccagggaaccctggtcaccgtctcctcagcctccggaggcggaggctccggtgggggaggatctgggggaggcggaagcgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaagagtctaagtacggaccgccctgccccccttgccctgctagcatgttctgggtgctggtggtggtcggaggcgtgctggcctgctacagcctgctggtcaccgtggccttcatcatcttttgggtccgcagcaagcggagcagaggcggccacagcgactacatgaacatgacccctagacggcctggccccaccagaaagcactaccagccctacgcccctccccgggactttgccgcctacagaagccgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgaacggaggcggggcaagggccacgacggcctgtatcagggcctgtccaccgccaccaaggatacctacgacgccctgcacatgcaggccctgcccccaaggtga

SEQ ID NO: 66 - protein, 463 aa, synthetic construct
MLLLVTSLLLCELPHPAFLLIPEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKESKYGPPCPPCPASMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 12 aa, synthetic construct
GGGSGGGSGGGS

(19) United States
(12) Patent Application Publication
(10) Pub. No.: US 2026/0048121 A1
(43) Pub. Date: Feb. 19, 2026

(54) EPITOPE ENGINEERING OF CD38 CELL-SURFACE RECEPTORS

(71) Applicants: DANA-FARBER CANCER INSTITUTE, INC., Boston, MA (US); THE CHILDREN'S MEDICAL CENTER CORPORATION, Boston, MA (US)

(72) Inventors: Pietro Genovese, Boston, MA (US); Gabriele Casirati, Boston, MA (US); Andrea Cosentino, Boston, MA (US)

(21) Appl. No.: 19/145,850

(22) PCT Filed: Jan. 5, 2024
(86) PCT No.: PCT/US2024/010421

§ 371 Date: Jul. 3, 2025

Related U.S. Application Data
(60) Provisional application No. 63/437,326, filed on Jan. 5, 2023.
(60) Provisional application No. 63/530,217, filed on Aug. 1, 2023.

(51) Int. Cl. A61K 40/11; A61K 40/31; A61K 40/42; C07K 14/725; C07K 16/28

(52) U.S. Cl. CPC A61K 40/11; A61K 40/31; A61K 40/4222; C07K 14/7051; C07K 16/2896

(57) ABSTRACT

Genetically engineered cells (e.g., HSPCs or T cells), such as hematopoietic stem cells, having one or more genetically edited genes of cell-surface proteins, and therapeutic uses thereof, either alone or in combination with immune therapy that targets the cell-surface protein(s).

### CROSS-REFERENCE TO RELATED APPLICATION

This application claims the benefit of U.S. Provisional Patent Application No. 63/437,326 filed Jan. 5, 2023, and U.S. Provisional Patent Application No. 63/530,217 filed Aug. 1, 2023, each of which is incorporated herein by reference in its entirety.

### BACKGROUND

Allogeneic hematopoietic stem/progenitor cell (HSPC) transplantation (HSCT) is currently at least used in clinical practice for the treatment of high-risk forms of acute leukemias or myelodysplastic syndromes but results in only 15-20% of long-term relapse free survival. Further, despite the recent successes of immunotherapies, their application for acute myeloid leukemia (AML) is hampered by the absence of leukemia-restricted targets. The most suitable candidates often have affinity for targets displayed by both diseased cells and healthy HSPCs. As such, the use of such candidates in AML therapy could result in immunosuppression and life-threatening hematopoietic toxicity. Ultimately, anti-myeloid/stem cell CAR-T-induced toxicity restricts applicability of these particular immunotherapeutics to a salvage therapy in a limited time window before HSCT, which may be insufficient for disease eradication.

Multiple myeloma (MM) is the second most common hematological malignancy in adults. Despite the approval of several new therapeutic agents which have extended patients' survival, MM remains largely incurable. Similar to AML, the development of immunotherapies for MM (e.g., CD38-targeted CAR-T) is limited by the fact that many surface targets are also widely expressed on hematopoietic cells.

There is a need for effective therapeutic agents that target cells of interest, such as cancer cells or host diseased hematopoietic stem cells. Most beneficially, such therapeutic agents would inflict minimal harm on normal cell populations.

### SUMMARY

The present disclosure generally relates to genetically engineered hematopoietic cells such as hematopoietic stem cells, progenitor cells, or T cells, having one or more genetically edited genes of cell-surface proteins, and chimeric antigen receptors that are capable of targeting the same cell-surface proteins. In certain embodiments, the genetically engineered cells are human hematopoietic stem cells (HSCs).

Provided herein is a genetically engineered hematopoietic stem/progenitor cell (HSPC) or T cell that include a genetically engineered CD38 gene, wherein the genetically engineered CD38 gene is engineered such that its encoded protein has reduced binding to a therapeutic anti-CD38 antibody (e.g., daratumumab). In some embodiments, the genetically engineered HSPC or T cell includes at least one mutation in the genetically engineered CD38 gene that results in a polypeptide bearing a mutation at position S274. In some embodiments, the mutation at position S274 is S274F. In some embodiments, the therapeutic anti-CD38 antibodis an antibody that has the same six complementarity-determining regions (CDRs) as daratumumab or is otherwise able to compete for CD38 binding sites with daratumumab.

Populations of such genetically engineered cells are also provided, as are compositions and kits that contain such cells.

The cells may be genetically engineered using a CRISPR system. The CRISPR system includes a guide nucleic acid, particularly guide RNAs, and a nuclease. The CRISPR system may be a base editing system that utilizes simple guide RNAs. Suitable polynucleotides are provided herein that function as guide RNAs for use in a base editing system.

In some embodiments of the CRISPR system used to form genetically modified genes, the nuclease is Streptococcus pyogenes Cas9 (SpCas9), Staphylococcus aureus (SaCas9), Lachnospiraceae bacterium Cas12a (LbCas12a), or Acidaminococcus sp. BV3L6 (AsCas12a). In some embodiments, the CRISPR system includes a SpCas9 nuclease. In some embodiments, the nuclease is a catalytically impaired SpCas9 nuclease linked to a base editor enzyme. In some embodiments, the base editor enzyme is a nucleotide deaminase. In some embodiments, the nucleotide deaminase is a cytidine deaminase or an adenosine deaminase.

Methods of treating a hematological condition (e.g., multiple myeloma, acute leukemia or a myelodysplastic syndrome or other lymphoid and myeloid malignancies) are also provided. Such methods include administering to a human subject: (a) a population of genetically engineered hematopoietic stem/progenitor cells or T cells as described herein, and (b) a therapeutically effective amount of at least one agent comprising an antibody binding domain or an antibody or antibody fragment comprising the antibody binding domain.

In some embodiments of the method of treating that uses genetically engineered cells that include a genetically engineered CD38 gene, the antibody is an anti-CD38 antibody. In some embodiments, the agent comprises a Chimeric Antigen Receptor-T (CAR-T) cell comprising an anti-CD38 binding domain. In some such embodiments, the hematological condition is multiple myeloma, acute leukemia or a myelodysplastic syndrome or other myeloid and lymphoid malignancies. Chimeric antigen receptors (CARs) comprising a polypeptide are also provided. In some embodiments, the polypeptide includes (a) one or more epitope binding fragments that binds to an epitope of one or more cell-surface lineage-specific proteins, (b) a hinge domain, (c) a transmembrane domain, (d) a co-stimulatory domain, and (e) a cytoplasmic signaling domain, wherein one of the cell-surface lineage-specific proteins is CD38.

Also provided herein are cells expressing any one of the CARs described herein. In some embodiments, the cell is an immune cell. In some embodiments, the immune cell is a T cell. Compositions and kits that contain such cells are also provided.

Also provided herein are methods of treating a hematological condition, particularly multiple myeloma, the method comprising administering to a human subject: (a) a population of genetically engineered hematopoietic stem/progenitor cells or T cells as described herein; and (b) a cell expressing any one of the CARs described herein.

Polypeptides formed from the genetically engineered genes described herein are also provided, as are nucleic acids encoding the polypeptides, vectors comprising the nucleic acids, and cells comprising the nucleic acid or vector. The present disclosure also provides a method of making a polypeptide, wherein the method comprises culturing cells under conditions that allow for the expression of the polypeptide, and optionally isolating the polypeptide.

### Definitions

Herein, the terms “identity” and “identical” are used to refer to sequence identity between two amino acid sequences or two nucleic acid sequences. The phrases “percent identity” and “percent identical” and simply “identity” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid (i.e., polynucleotide) sequences.

Methods of alignment of sequences for comparison are well known in the art. Typically, one sequence acts as a reference sequence, to which test sequences are compared by aligning the residues of the two sequences (for example, a candidate polypeptide or polynucleotide and the reference polypeptide or polynucleotide of a specific sequence) to optimize the number of identical amino acids or nucleotides along the lengths of their sequences. Gaps in either or both sequences are permitted in making the alignment in order to optimize the number of identical amino acids, although the amino acids or nucleotides in each sequence must nonetheless remain in their proper order. The amino acid residues or nucleotides at corresponding amino acid positions or nucleotide positions are then compared. For example, a pair-wise comparison analysis of sequences can be carried out using the BESTFIT algorithm in the GCG package (version 10.2, Madison WI). Alternatively, sequences may be compared using the Blastp program of the BLAST 2 search algorithm, as described by Tatiana et al., (FEMS Microbiol. Lett., 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

As used herein, the term “epitope” refers to an amino acid sequence (linear or conformational) of a protein, such as a cell-surface antigen, that is bound by the complementarity determining regions (CDRs) of an antibody.

As used herein, “subject,” “individual,” and “patient” are used interchangeably, and refer to a human.

As used herein, the term “effective amount” can be used interchangeably with the term “therapeutically effective amount.” and refers to that quantity of a cytotoxic agent, genetically engineered cell population, or pharmaceutical composition (e.g., a composition comprising cytotoxic agents and/or genetically engineered cells) that is sufficient to result in a desired activity, such as to delay the manifestation, arrest the progression, or improve, relieve, reduce, ameliorate, or alleviate at least one symptom, of a disorder upon administration to a subject in need thereof.

Herein, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained. At the very least, and not as an attempt to limit the doctrine of equivalents to the scope of the claims, each numerical parameter should at least be construed in light of the number of reported significant digits and by applying ordinary rounding techniques.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

In the preceding description, particular embodiments may be described in isolation for clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The polynucleotide sequences described herein are described using DNA or RNA. It is understood that the complements, reverse sequences, and reverse complements of the DNA and RNA sequences can be easily determined by the skilled person and are within the scope of the present disclosure. It is also understood that the sequences disclosed herein as DNA sequences can be converted from a DNA sequence to an RNA sequence by replacing each thymidine nucleotide (T) with a uridine nucleotide (U). When a DNA sequence is used to describe RNA (e.g., guide RNA), it is understood that the corresponding RNA sequence is the DNA sequence with each thymidine nucleotide (T) or thymine nucleobase replaced with a uridine nucleotide (U) or uracil nucleobase. For example, a guide RNA of the DNA sequence GCGTATAG has an RNA sequence of GCGUAUAG. The nucleotide being replaced, or the replacement nucleotide may be modified (e.g., a modified sugar, a modified internucleoside linkage, and the like).

Polynucleotide and/or polypeptides or protein sequences may include one or more forms of typographical emphasis (e.g., underlined text, bolded text, italicized text). It is understood that the typographical emphasis is non-limiting. Sequences stated with typographical emphasis include the stated sequence without the typographical emphasis. Typographical emphasis may or may not indicate a modified nucleotide base or linkage; a modified sequence relative to an indicated sequence; the location of a feature such as spacer, particular codon or codons, particular amino acid or amino acids, primer binding site, a mutation site, a retrotranscriptase template, a complementarity-determining region, or the like; or any combination thereof Additionally, polynucleotide sequences may be displayed in capital letters, lower case letters, or a combination thereof. Although the case of the letters in the polynucleotide sequences may be used to distinguish portions of a sequence, the case of the letters is non-limiting. Unless otherwise stated, lower case and upper case letters indicate the identity of the nucleobase.

The above summary is not intended to describe each disclosed embodiment or every implementation thereof. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

Unless otherwise defined, all technical and scientific terms used herein have the same meaning as commonly understood by one of ordinary skill in the corresponding art. Methods and materials are described herein; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description, figures, and claims.

### BRIEF DESCRIPTION OF THE DRAWINGS

FIG. 1A is a crystal structure showing that one orthologous mutation from Macaca Fascicularis to the human CD38 protein can efficiently impair the binding of the antibody Daratumumab on CD38 glycoprotein (reproduced from protein data bank number 7dha).

FIG. 1B is a binding curve showing that mutation of S274F impairs the binding of the antibody daratumumab on CD38 glycoprotein, whereas binding of other competition group 3 Abs was not affected (EC₅₀ values: HM-025, 15 nanograms per milliliter (ng/mL); HM-028, 15 nanograms per liter (ng/L); HM-034, 17 ng/mL) (reproduced from Michel de Weers et al. in J. Immunol., 186 (3): 1840-1848 (2011).

FIG. 1C is a schematic showing genetic engineering of CD38 (top) and the gRNA used (bottom).

FIGS. 1D, 1E, and 1F are various Fluorescence Activated Cell Sorting (FACS) analyses showing that the K562 cells were genetically modified.

FIG. 2A is a schematic design of daratumumab based anti-CD38 CAR-1 and anti-CD38 CAR-2.

FIGS. 2B, 2C, and 2D show T cells were activated through anti-CD3/CD28 beads (FIG. 2A) and cultured in IMDM plus IL7-IL15 (FIG. 2B). The FACS histogram (FIG. 2D) shows the expression of CD38 marker at day 3 (D3) of stimulation (before CAR transduction).

FIGS. 2E, 2F, 2G, 2H, and 2I show expression of CD38 at day 7 (D7) of stimulation on different CAR-T cells (Anti-CD38 CAR-1 (FIG. 2E), Anti-CD38 CAR-2 (FIG. 2F), Anti cKIT CAR-1 (FIG. 2G) and Anti cKIT CAR-2 (FIG. 2H)). Both the anti-CD38 CAR-T cells lost the expression of CD38 while the anti-cKIT CAR-T cells retained CD38 expression. An untransduced control (UT) was included (FIG. 2I).

FIG. 2J shows the lack of expansion of the anti-CD38 CAR-T cells (bottom two lines), while anti-cKIT CAR-T cells (produced as a positive control) shows a fold expansion which is comparable to the untransduced control (UT).

FIG. 2K shows the enrichment of CD38 knock out (KO) T cells in the presence of anti CD38-CAR that is compatible with a fratricide effect on the CD38 expressing T cells. The KO was performed using the CD38 gRNA in combination with a Cas9 protein.

FIG. 3A shows the percentage of live target cells (K562 overexpressing CD38) after 4 hours of co-culture with untransduced (UT) or anti-CD38-CAR-T cells, at different effector:target ratio. The readout was obtained through Flow Cytometry after an apoptosis staining (7AAD-Annexin).

FIG. 3B shows the percentage of live target cells K562 wild-type (WT) (negative for CD38) after 4 hours of co-culture with anti CD38-CAR-T cells, at different effector:target ratio. The readout was obtained through Flow Cytometry after an apoptosis staining (7AAD-Annexin).

### DETAILED DESCRIPTION

Identifying suitable proteins for targeted cancer therapies presents a significant challenge. Many potential target proteins are present on both the cell surface of a cancer cell and on the cell surface of normal, non-cancer cells, which can be involved in the development and/or survival of the subject. Many of the target proteins contribute to the functionality of such cells. Thus, therapies targeting these proteins can lead to deleterious effects in the subject, such as significant toxicity and/or other side effects. Further, resistance to chimeric antigen receptor T cell (CAR-T) therapy remains a challenge in treatment of hematopoietic malignancies, such as acute myeloid leukemia (AML) and multiple myeloma (MM), due to switch of cancer antigens on cancer cells, thereby escaping CAR-T therapy. In one aspect of the present disclosure, the replacement of cancer cells by a modified population of normal cells is performed using normal cells that have been manipulated such that the cells do not bind a cytotoxic agent.

Accordingly, the present disclosure provides methods, cells, compositions, and kits. The methods, cells, compositions, and kits described herein may be administered to a subject suffering from a hematological condition (e.g., a malignancy). The methods, cells, compositions, and kits described herein may provide an effective treatment for hematological conditions, particularly malignancies, allowing for targeting of one or more cell surface proteins that are present not only on cancer cells but also on cells critical for the development and/or survival of the subject. In some instances, described herein are genetically engineered cells (e.g., HSPCs or T cells) such as hematopoietic stem/progenitor cells (HSPCs) having genetic editing in one or more genes coding for cell-surface proteins, for example, CD38; methods of producing such, for examples, using a nucleotide-guided gene editor (CRISPR) approach with specific guide RNAs; methods of treating a hematopoietic condition, particularly a malignancy, using the engineered hematopoietic cells, either taken alone, or in combination with one or more cytotoxic agents (e.g., CAR-T cells) that can target the wild-type cell-surface antigens but not those encoded by the edited genes in the engineered hematopoietic cells; and kits comprising the engineered hematopoietic cells.

I. Genetically Engineered Cells (e.g., HSPCs and/or T cells)

In some embodiments, the genetically engineered cells (e.g., HSPCs or T cells) have an edited CD38 gene. In some embodiments, one or more of these genes are mutated. In some instances, the mutated CD38 gene includes mutations or deletions in one or more non-essential epitopes so as to retain (in whole or in part) the bioactivity of the CD38 gene.

i. Hematopoietic Stem/Progenitor Cells (HSPCs)

In some embodiments, the hematopoietic cells described herein are hematopoietic stem/progenitor cells. Hematopoietic stem/progenitor cells (HSPCs) are capable of giving rise to both myeloid and lymphoid progenitor cells that further give rise to myeloid cells (e.g., monocytes, macrophages, neutrophils, basophils, dendritic cells, erythrocytes, platelets, and the like.) and lymphoid cells (e.g., T cells, B cells, NK cells), respectively. HSPCs are characterized by the expression of the cell surface marker CD34 (e.g., CD34+), which can be used for the identification and/or isolation of HSPCs.

In some embodiments, the HSPCs are obtained from a human subject. In some embodiments, the human subject is a non-human primate, a rodent (e.g., mouse or rat), a bovine, a porcine, an equine, or a domestic animal. In some embodiments, the HSPCs are obtained from a human patient, such as a human patient suffering from a hematopoietic malignancy. In some embodiments, the HSPCs are obtained from a healthy donor. In some such embodiment, the HSPCs are obtained from a donor not suffering from a hematopoietic malignancy. In some embodiments, the HSPCs are obtained from the subject to whom the genetically engineered HSPCs will be subsequently administered. HSPCs that are administered to the same subject from which the cells were obtained are referred to as autologous cells. HSPCs that are obtained from a subject who is not the subject to whom the cells will be administered are referred to as allogeneic cells. In embodiments wherein the cells are allogeneic cells, the method may be modified to reduce incidence of rejection. Methods to reduce incidence of rejection are standard and well known in the art.

HSPCs can be obtained from any suitable source using conventional means known in the art. In some embodiments, HSPCs are obtained from a sample from a subject (or donor), such as bone marrow, blood (e.g., peripheral blood mononuclear cells (PBMCs), and/or an umbilical cord (i.e., cord blood cells). In general, bone marrow cells can be obtained from iliac crest, femora, tibiae, spine, rib or other medullary spaces of a subject (or donor). Bone marrow can be taken out of the patient and isolated through various separations and washing procedures known in the art.

HSPCs typically reside in the bone marrow but can be mobilized into the circulating blood by administering a mobilizing agent in order to harvest HSPCs from the peripheral blood. In some embodiments, the subject (or donor) from which the HSPCs are obtained is administered a mobilizing agent, such as granulocyte colony-stimulating factor (G-CSF). The number of the HSPCs collected following mobilization using a mobilizing agent is typically greater than the number of cells obtained without use of a mobilizing agent.

In some embodiments, a sample is obtained from a subject (or donor) and is then enriched for a desired cell type (e.g., CD34+, CD34+CD38−, CD133+, CD90+, CD49f+). For example, PBMCs and/or CD34+ hematopoietic cells can be isolated from blood. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement. ii. T cells

T cells are typically immune cells of the lymphoid lineage. T cells express the T cell receptor (TCR), with most cells expressing alpha and beta chains and a smaller population expressing gamma and delta chains. T cells useful as immune cells can be CD4+ or CD8+ and can include, but are not limited to, T helper cells (CD4+), cytotoxic T cells (also referred to as cytotoxic T lymphocytes, CTL; CD8− T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, T regulatory cells, natural killer T cells, mucosal associated invariant T cells (MAIT), and gamma/delta T cells. Major T cell subtypes include TN (naive), TSCM (stem cell memory), TCM (central memory), TTM (Transitional Memory), TEM (effector memory), and TTE (terminal effector). In one embodiment, the T cells are immunostimulatory cells, i.e., cells that mediate an immune response. Exemplary T cells that are immunostimulatory include, but are not limited to, T helper cells (CD4+), cytotoxic T cells (also referred to as cytotoxic T lymphocytes, CTL; CD8+ T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, natural killer T cells, mucosal associated invariant T cells (MAIT), and gamma/delta T cells. In another embodiment, the T cells are immunoinhibitory cells, i.e., cells that inhibit an immune response. Exemplary T cells that are immunoinhibitory include regulatory T cells (T regulatory cells, Treg) and follicular regulatory T cells (Tfh) cells. T cells can optionally be generated from embryonic stem cells or induced pluripotent stem cells (iPSCs) (see, for example, Themeli et al., Nat. Biotechnol. 31(10):928-933 (2013)).

The type of T cell selected typically depends on whether it is desired to stimulate an immune response or inhibit an immune response. For example, a regulatory T cell (CD4+CD25high FoxpP3+) would be used for treating a subject in need of an inhibited immune response such as someone having an autoimmune disease, while CD4+(except Treg)/CD8+ T cells are used to treat a subject in need of a stimulated immune response, for example, a subject having cancer.

T cells can be isolated by methods well known in the art, including commercially available isolation methods. Sources for the T cells include, but are not limited to, peripheral blood, umbilical cord blood, bone marrow, or other sources of hematopoietic cells. Various techniques can be employed to separate the cells to isolate or enrich for desired immune cells such as T cells. For instance, negative selection methods can be used to remove cells that are not the desired immune cells. Additionally, positive selection methods can be used to isolate or enrich for desired T cells, or a combination of positive and negative selection methods can be employed. Monoclonal antibodies (MAbs) are particularly useful for identifying markers associated with particular cell lineages and/or stages of differentiation for both positive and negative selections. If a particular type of T cell is to be isolated, various cell surface markers or combinations of markers, including but not limited to, CD3, CD4, CD8, CD34 (for hematopoietic stem and progenitor cells) and the like, can be used to separate the cells, as is well known in the art.

The T cells can be autologous or non-autologous to the subject to which they are administered in the methods of treatment disclosed herein. Optionally, the cells can be obtained by leukapheresis, where leukocytes are selectively removed from withdrawn blood, made recombinant, and then transfused back into the donor. Alternatively, allogeneic cells from a non-autologous donor other than the subject can be used. In the case of a non-autologous donor, the cells are typed and matched for human leukocyte antigen (HLA) to determine an appropriate level of compatibility, as is well known in the art. For both autologous and non-autologous cells, the cells can optionally be cryopreserved until ready to be used for genetic manipulation and/or administration to a subject using methods well known in the art. iii. Mutated Cell-Surface Antigens

In some embodiments, the hematopoietic stem/progenitor cells (HSPCs) or T cells described herein can contain an edited gene encoding one or more cell-surface proteins of interest (e.g., CD38) in mutated form (mutants or variants, which are used herein interchangeably). The mutant can have reduced binding or no binding to a cytotoxic agent as described herein (e.g., anti-CD38 antibody). The mutants can include one or more mutations of the epitope (e.g., the nucleotide sequence encoding the epitope and the amino acid sequence of the epitope) to which the cytotoxic agent binds, such that binding to the cytotoxic agent is reduced or abolished as compared to the natural or wild-type cell-surface protein counterpart. Such a mutant may be preferred to maintain substantially similar biological activity as the wild-type counterpart.

As used herein, the term “reduced binding” refers to binding that is reduced by at least 25%. The level of binding can refer to the amount of binding of the cytotoxic agent to a hematopoietic stem cell, progenitor cell, or to a T cell, or the amount of binding of the cytotoxic agent to the cell-surface protein as compared to a wild-type (i.e., non-engineered, non-mutated) protein. In some embodiments, the binding is reduced by at least 25%, at least 30%, at least 40%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In some embodiments, the binding is reduced such that there is substantially no detectable binding in a conventional assay. As used herein, “no binding” refers to substantially no binding, e.g., no detectable binding or only baseline binding as determined in a conventional binding assay. Binding and reduced binding can be measured using quantitative fluorescence reduction, for example, by conducting a fluorescence activated cell sorting (FACS) titration.

In some embodiments, the variant (mutant) contains one or more amino acid residue substitutions (e.g., 1, 2, 3, 4, 5, or more) within the epitope of interest such that the cytotoxic agent does not bind or has reduced binding to the mutated epitope. Such a mutant can have substantially reduced binding affinity to the cytotoxic agent (e.g., having a binding affinity that is at least 40%, at least 50%, at least 60%, at least 70%, at least 80%, or at least 90% lower than its wild-type counterpart) or abolished binding activity to the cytotoxic agent. In other instances, the mutant contains a deletion of a region that includes the epitope of interest. Such a region can be encoded by an exon. In some embodiments, the region is a domain of the cell-surface protein of interest that encodes the epitope. In one example, the variant has just the epitope deleted. The length of the deleted region can range from 3-60 amino acids, for example, 5 to 50, 5 to 40, 10 to 30, 10 to 20, 5 to 10, and the like.

In some embodiments, the cytotoxic agent binds to one or more (e.g., at least 2, at least 3, at least 4, at least 5, or more) epitopes of a cell-surface antigen. In some embodiments, the cytotoxic agent binds to more than one epitope of the cell-surface antigen and the cells (e.g., HSPCs or T cells) are manipulated such that each of the epitopes is absent and/or unavailable for binding by the cytotoxic agent.

The mutation(s) or deletions in a mutant of a cell-surface antigen can be within or around a non-essential epitope such that the mutation(s) or deletion(s) do not substantially affect the bioactivity of the protein.

In some embodiments, the genetically engineered cells (e.g., HSPCs or T cells) described herein have one or more edited genes of cell-surface antigens such that the edited genes express mutated cell-surface antigens with mutations in one or more non-essential epitopes. A “non-essential epitope” (or a fragment comprising such) refers to a domain within the cell surface protein/antigen, a mutation to which is less likely to substantially affect the bioactivity of the cell surface protein. For example, when the engineered cells (e.g., HSPCs or T cells) comprise a deletion or mutation of a non-essential epitope of a cell-surface antigen, such engineered cells are able to proliferate and/or undergo erythropoietic differentiation to a similar level as cells that express a wild-type cell-surface antigen. Methods for identifying and/or verifying non-essential epitopes in cell-surface antigens are well known. Further, methods for assessing the functionality of the cell-surface antigen and the engineered cells are known in the art and include, for example, proliferation assays, differentiation assays, colony formation assays, expression analysis (e.g., gene and/or protein), protein localization assays, intracellular signaling assays, functional assays, and the study of humanized mouse models.

iv. Preparation of Genetically Engineered Cells (e.g., HSPCs or T Cells)

Any of the genetically engineered cells (e.g., HSPCs or T cells), that include edited genes encoding one or more cell-surface antigens can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to alter the expression of a target gene. In general, genome editing methods involve use of an endonuclease that is capable of cleaving the nucleic acid of the genome. For example, an endonuclease may cleave the nucleic acid sequence of the genome at a targeted nucleotide sequence. In some instances, genome editing methods involve use of a catalytically “dead” nuclease or a nuclease that is a nickase. Repair of double-stranded breaks in the genome often introduces mutations and/or introduces exogenous nucleic acid into the targeted site. In some cases, genome editing methods involve use of a catalytically inactive or partially inactive endonuclease fused to a functional domain, e.g., an adenine or cytidine deaminase domain in the case of base editors. Other functional domains include reverse transcriptases, RNA-binding proteins, transcription factors, DNA repair machinery, prime editors, CRISPR-Cas activators or repressors, and the like.

Genome editing methods are generally classified based on the type of endonuclease that is involved in generating double stranded breaks in the target nucleic acid. Types of genome editing methods include use of zinc finger nucleases (ZFN), transcription activator-like effector-based nuclease (TALEN), meganucleases, and Clustered Regularly Interspaced Short Palindromic Repeats (CRISPR) and CRISPR-associated (Cas) protein systems. Modification (editing) can include the deletion or mutation of an epitope of the specific cell-surface protein using a CRISPR/Cas system, such as CRISPR/Cas9.

v. CRISPR-Cas System

In some embodiments, the genetically engineered HSPCs are genetically engineered using a CRISPR system. A CRISPR system includes a guide nucleic acid and a nuclease. Cas nucleases can be readily programmed to cleave target DNA sequences for genome editing in various organisms. One class of these nucleases, referred to as Cas9 proteins or Cas9 nucleases, form a complex with two short RNAs: a crRNA and a trans-activating crRNA (tracrRNA). The crRNA and tracrRNA typically hybridize to form a guide RNA (gRNA). The most commonly used Cas9 ortholog, S. pyogenes cas9 (SpCas9), uses a crRNA that has a 20 nucleotide (nt) “spacer” region at its 5′ end that is complementary to the strand opposite the “protospacer” region of the target DNA site. Efficient cleavage includes SpCas9 recognizing a protospacer adjacent motif (PAM). The crRNA and tracrRNA sequences may be joined to form a single approximately100-nt single guide RNA (sgRNA, a type of gRNA) that directs the DNA cleavage activity of SpCas9. A Cas protein named Cpf1 (also called Cas12a) has been identified that can also be programmed to cleave target DNA sequences. Unlike SpCas9, Cpf1 does not include a tracrRNA sequence, but instead uses a single 42-nt crRNA, which has 23-nt at its 3′ end that are complementary to the protospacer of the target DNA sequence.

In some embodiments, the Cas endonuclease is a Cas9 nuclease or variant thereof, which cleaves both strands of the double stranded DNA of a target nucleic acid resulting in blunt ends. In some embodiments, the Cas endonuclease is a Cpf1 nuclease or variant thereof, which results in cleaves both strands of the double-stranded DNA of a target nucleic acid resulting in staggered ends of the nucleic acid.

### CRISPR Cas9 System

In some embodiments, the Cas endonuclease is a Cas9 enzyme or variant thereof. In some embodiments, the Cas9 endonuclease is derived from Streptococcus pyogenes (SpCas9) having a known (wild-type) sequence (see uniprot.org/uniprotkb/Q99ZW2/entry, Accession No. AAK33936.1), or has a sequence that is at least 80%, at least 85%, at least 90%, or at least 95% identical to the amino acid sequence of the wild-type SpCas9 endonuclease, e.g., having differences at up to 5%, up to 10%, up to 15%, or up to 20% of the residues replaced, e.g., with conservative mutations. In some embodiments, the Cas9 endonuclease is derived from Staphylococcus aureus (SaCas9) having a known (wild-type) sequence (see uniprot.org/uniprotkb/J7RUA5/entry; Accession No. CCK74173.1), or has a sequence that is at least 80%, at least 85%, at least 90%, or at least 95% identical to the amino acid sequence of the wild-type SaCas9, e.g., having differences at up to 5%, up to 10%, up to 15%, or up to 20% of the residues replaced, e.g., with conservative mutations. In preferred embodiments, the endonuclease retains desired activity of the parent, e.g., the nuclease activity (except where the parent is a nickase or a dead Cas9), and/or the ability to interact with a guide RNA and target DNA).

Herein, in the context of amino acid sequences, a “conservative” mutation (i.e., conservative substitution) for an amino acid in an endonuclease or other polypeptide described herein may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, or glutamine. The positively charged (basic) amino acids include arginine, lysine, or histidine. The negatively charged (acidic) amino acids include aspartic acid or glutamic acid. Conservative substitutions include, for example, Lys for Arg or vice versa to maintain a positive charge; Glu for Asp or vice versa to maintain a negative charge; Ser for Thr or vice versa so that a free —OH is maintained; or Gln for Asn or vice versa to maintain a free —NH₂. Likewise, biologically active analogs of a polypeptide containing deletions or additions of one or more contiguous or noncontiguous amino acids that do not eliminate a functional activity of the polypeptide are also contemplated.

In general, the target nucleic acid is flanked on the 3′ side or 5′ side by a protospacer adjacent motif (PAM) that can interact with the endonuclease and be further involved in targeting the endonuclease activity to the target nucleic acid. It is generally thought that the PAM sequence flanking the target nucleic acid depends at least in part on the endonuclease and the source from which the endonuclease is derived. For example, for Cas9 endonucleases that are derived from Streptococcus pyogenes, the PAM sequence is NGG, although the PAM sequences NAG and NGA can be recognized with lower efficiency ((N is A, C, G, or T). For Cas9 endonucleases derived from Staphylococcus aureus, the PAM sequence is NNGRRT (N is A, C, G, or T; R is A or G).

Accordingly, in some embodiments, the endonuclease is engineered/modified such that it can recognize one or more PAM sequences. In some embodiments, the endonuclease has been engineered/modified to recognize one or more PAM sequence that is different than the PAM sequence the endonuclease recognizes without engineering/modification. In some embodiments, the endonuclease may be modified such that it can recognize a PAM sequence lacking a guanine. In some embodiments, the endonuclease may be modified such that it can recognize a PAM sequence including “ACA,” “AGC,” or “AAA.” In some embodiments, the endonuclease has been engineered/modified to reduce off-target activity of the enzyme. In some embodiments, the nucleotide sequence encoding the endonuclease is modified to alter the PAM recognition of the endonuclease. For example, the Cas endonuclease (e.g., SpCas9) has mutations at one or more of the following positions: A61, L1111, D1135, S1136, G1218, E1219, N1317, A1322, R1333, R1335, T1337. See, for example, International Patent Application Publication Nos. WO 2016/141224 and WO 2017/040348, US Patent Application Publication No. 2021/0284978A1.

In some embodiments, the Cas9 endonuclease is a catalytically inactive (i.e., catalytically impaired) Cas9. For example, dCas9 contains mutations at catalytically active residues (D10, E762, D839, H983, or D986; and/or at H840 or N863) and does not have nuclease activity. For example, the mutations are: (i) D10A or D10N, and/or (ii) H840A, H840N, or H840Y. In some embodiments, the catalytically impaired SpCas9 includes a mutation at position D10A. In some embodiments, the catalytically impaired SpCas9 includes the mutationD10N. In some embodiments, the catalytically impaired SpCas9 includes a mutation at position K918. In one or more embodiments, the catalytically impaired SpCas9 includes the mutation K918N.

In some embodiments, the nucleotide sequence encoding the Cas9 endonuclease is further modified to alter the activity of the protein. In some embodiments, the Cas9 endonuclease has been modified to inactivate one or more catalytic residues of the endonuclease. In some embodiments, the Cas9 endonuclease has been modified to inactivate one of the catalytic residues of the endonuclease, referred to as a “nickase” or “Cas9n.” Cas9 nickase endonucleases cleave one DNA strand of the target nucleic acid.

In some embodiments, the catalytically impaired SpCas9 is NG-SpCas9 or SpRY-SpCas9. The endonuclease NG-SpCas9 nickase has the following mutations relative to wild-type SpCas0: D10A, L1111R, D1135V, G1218R, E1219F, A1322R, R1335V, and T1337R. The endonuclease SpRY-Cas9 nickase has the following mutations relative to wild-type SpCas9: D10A, A61R, L1111R, D135L, S1136W, G1218K, E1219Q, N1317R, A1322R, R1333P, R1335Q, and T1337R.

### CRISPR Cpf1 (Cas12a)

In some embodiments, the Cas endonuclease is a Cpf1 nuclease (also referred to as Cas12a) or variant thereof. Cpf1 endonuclease generally recognize a PAM sequence located at the 5′ end of the target nucleic acid. For a Cpf1 nuclease, the PAM sequence is TTTN (N is A, C, G, or T). In some embodiments, the host cell expresses a Cpf1 nuclease derived from Lachnospiraceae bacterium (LbCpf1), Acidaminococcus sp. (AsCpf1), or Francisella tularensis (FnCpf1). Wild-type sequences for each are known: Type V CRISPR-associated protein Cpf1 (Lachnospiraceae bacterium ND2006), GenBank Acc No. WP_051666128.1; Type V CRISPR-associated protein Cpf1 [Acidaminococcus sp. BV3L6], NCBI Reference Sequence: WP_021736722.1; Type V CRISPR-associated protein Cpf1 (Francisella tularensis), GenBank Acc No. WP_003040289.1.

In some embodiments, the Cpf1 endonuclease is the wild-type version of the nuclease. In some embodiments, the Cpf1 endonuclease is at least 80%, at least 85%, at least 90%, or at least 95% identical to the amino acid sequence of the wild-type sequence, e.g., having up to 5%, up to 10%, up to 15%, or up to 20% of the residues replaced, e.g., with conservative mutations. In some embodiments, the endonuclease retains desired activity of the parent, e.g., the nuclease activity (except where the parent is a nickase or a dead Cas9), and/or the ability to interact with a guide RNA and target DNA).

In some embodiments, the Cas12a endonuclease is a catalytically inactive variant, which can be referred to dCas12a.

### Cas Endonuclease Functional Domains and CRISPR Base Editing System

Alternatively, or in addition, the Cas endonuclease (i.e., Cas9 or Cas12a) can be fused to another protein or portion thereof, e.g., a heterologous functional domain. In some embodiments, the heterologous functional domain is a transcriptional activation domain (e.g., VP64 or NF-KB p65). In some embodiments, the heterologous functional domain is a transcriptional silencer or transcriptional repression domain (e.g., wherein the transcriptional repression domain is Kruppel-associated box (KRAB) domain, ERF repressor domain (ERD), or mSin3A interaction domain (SID); wherein the transcriptional silencer is Heterochromatin Protein 1 (HIP1)). In some embodiments, the heterologous functional domain is an enzyme that modifies the methylation state of DNA (e.g., a DNA methyltransferase (DNMT) or a TET protein (such as, TETI)). In some embodiments, the heterologous functional domain is an enzyme that modifies a histone subunit (e.g., a histone acetyltransferase (HAT), histone deacetylase (HDAC), histone methyltransferase (HMT), or histone demethylase). In some embodiments, the heterologous functional domain is a biological tether (e.g., MS2, Csy4 or lambda N). In some embodiments, the heterologous functional domain is FokI.

In some embodiments, the heterologous functional domain and the endonuclease form a base editor. In some such embodiments, the heterologous functional domain may be such as a deaminase that modifies cytosine DNA bases, e.g., a cytidine deaminase from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, APOBEC4, activation-induced cytidine deaminase (AID), cytosine deaminase 1 (CDA1), and CDA2, and cytosine deaminase acting on tRNA (CDAT). Specific examples of base editors include evoAPOBEC1-BE4max, eA3A-BE5, EA-BE4max, or the deaminase disclosed in Neugebauer, Monica, et al., Nat. Biotechnol. 1-13 (2022) and Nat. Biotechnol., 41, 673-685 (2023).

In some embodiments, the heterologous functional domain is a deaminase that modifies adenosine DNA bases, e.g., the deaminase is an adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA). For example, ABE8e-TadA-8e. In some embodiments, the TadA adenosine deaminase domain includes a V106W mutation.

In some embodiments, the endonuclease is a base editor. Base editor endonucleases generally include a catalytically inactive Cas endonuclease fused to a base editor. For example, the endonuclease is SpCas9 with a mutation at D10, E762, D839, H983, or D986; and/or at H840 or N863 and fused to a base editor, such as those mentioned above.

Base editors can be used in CRISPR base editing methodologies that can directly install point-mutations in cellular DNA without inducing a double-strand DNA break. For example, a cytosine base editor is targeted to a specific locus by a guide RNA, and converts cytidine to uridine, which is then converted to thymidine through base excision repair, creating a C to T change (or a G to A on the opposite strand). An adenine base editor converts adenosine to inosine, which is treated like guanosine by the cell, creating an A to G (or T to C) change. Generally, base editing technology edits target nucleotides without creating double-strand breaks or relying on homology-directed repair. Such systems are commercially available (e.g., at addgene.org) and are described, for example, in AC Komor et al., Nature, 533: 420-424 (2016).

In some embodiments, the heterologous functional domain is an enzyme, domain, or peptide that inhibits or enhances endogenous DNA repair or base excision repair (BER) pathways, e.g., uracil DNA glycosylase inhibitor (UGI) that inhibits uracil DNA glycosylase (UDG, also known as uracil N-glycosylase, or UNG) mediated excision of uracil to initiate BER; or DNA end-binding proteins such as Gam from the bacteriophage Mu.

In some instances, the endonuclease (Cas9 or Cas12a) is fused to one or more of a nuclear localization sequence, cell penetrating peptide sequence, affinity tag, and/or a fluorescent protein. For example, the nuclear localization sequence is the SV40 large T-antigen nuclear localization sequence (PKKKRKV; SEQ ID NO: 1), the nucleoplasmin nuclear localization sequence (KRPAATKKAGQAKKKK; SEQ ID NO: 2) or the c-Myc nuclear localization sequence (PAAKRVKLD; SEQ ID NO: 3). For example, the nuclear localization sequence(s) is fused to the N-terminus and/or to the C-terminus of the Cas9 or Cas12a protein. In some embodiments, when a heterologous functional domain is fused to the N-terminus and/or to the C-terminus of the Cas9 or Cas12a protein, the nuclear localization sequence(s) is fused to the N-terminus and/or to the C-terminus of the heterologous functional domain-Cas protein complex or interposed between the heterologous functional domain and the Cas protein.

Sequences of exemplary Cas endonucleases are provided below:

SEQ ID NO: 4-Amino acid sequence of the SpRY-ABE8e-V106W 3xNLS adenine base editor:
MKRTADGSEFESPKKKRKVSEVEFSHEYWMRHALTLAKRARDEREVPVGAVLVLNNR
VIGEGWNRAIGLHDPTAHAEIMALRQGGLVMQNYRLIDATLYVTFEPCVMCAGAMIHS
RIGRVVFGWRNSKRGAAGSLMNVLNYPGMNHRVEITEGILADECAALLCDFYRMPRQV
FNAQKKAQSSINSGGSSGGSSGSETPGTSESATPESSGGSSGGSDKKYSIGLAIGTNSVGW
AVITDEYKVPSKKFKVLGNTDRHSIKKNLIGALLFDSGETAERTRLKRTARRRYTRRKN
RICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLS
MPQVNIVKKTEVQTGGFSKESIRPKRNSDKLIARKKDWDPKKYGGFLWPTVAYSVLVV
AKVEKGKSKKLKSVKELLGITIMERSSFEKNPIDFLEAKGYKEVKKDLIIKLPKYSLFELE
NGRKRMLASAKQLQKGNELALPSKYVNFLYLASHYEKLKGSPEDNEQKQLFVEQHKH
YLDEIIEQISEFSKRVILADANLDKVLSAYNKHRDKPIREQAENIIHLFTLTRLGAPRAFKY
FDTTIDPKQYRSTKEVLDATLIHQSITGLYETRIDLSQLGGDSGGSKRTADGSEFEPKKKR
KVGSGSKRPAATKKAGQAKKKKLE
SEQ ID NO: 5-Amino acid sequence of the SpRY-ABE8e 3xNLS adenine base editor:
MKRTADGSEFESPKKKRKVSEVEFSHEYWMRHALTLAKRARDEREVPVGAVLVLNNR
VIGEGWNRAIGLHDPTAHAEIMALRQGGLVMQNYRLIDATLYVTFEPCWMCAGAMIHS
RIGRVVFGWRNSKRGAAGSLMNVLNYPGMNHRVEITEGILADECAALLCDFYRMPRQV
FNAQKKAQSSINSGGSSGGSSGSETPGTSESATPESSGGSSGGSDKKYSIGLAIGTNSVGW
AVITDEYKVPSKKFKVLGNTDRHSIKKNLIGALLFDSGETAERTRLKRTARRRYTRRKN
RICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLS
MPQVNIVKKTEVQTGGFSKESIRPKRNSDKLIARKKDWDPKKYGGFLWPTVAYSVLVV
AKVEKGKSKKLKSVKELLGITIMERSSFEKNPIDFLEAKGYKEVKKDLIIKLPKYSLFELE
NGRKRMLASAKQLQKGNELALPSKYVNFLYLASHYEKLKGSPEDNEQKQLFVEQHKH
YLDEIIEQISEFSKRVILADANLDKVLSAYNKHRDKPIREQAENIIHLFTLTRLGAPRAFKY
FDTTIDPKQYRSTKEVLDATLIHQSITGLYETRIDLSQLGGDSGGSKRTADGSEFEPKKKR
KVGSGSKRPAATKKAGQAKKKKLE
SEQ ID NO: 6-Amino acid sequence of the SpRY-evoAPOBEC1-BE4 3xNLS adenine base editor:
MKRTADGSEFESPKKKRKVSSKTGPVAVDPTLRRRIEPHEFEVFFDPRELRKETCLLYEI
NWGGRHSIWRHTSQNTNKHVEVNFIEKFTTERYFCPNTRCSITWFLSWSPCGECSRAITE
FLSRYPNVTLFIYIARLYHLANPRNRQGLRDLISSGVTIQIMTEQESGYCWHNFVNYSPSN
ESHWPRYPHLWVRLYVLELYCIILGLPPCLNILRRKQSQLTSFTIALQSCHYQRLPPHILW
ATGLKSGGSSGGSSGSETPGTSESATPESSGGSSGGSDKKYSIGLAIGTNSVGWAVITDEY
KVPSKKFKVLGNTDRHSIKKNLIGALLFDSGETAERTRLKRTARRRYTRRKNRICYLQEI
FSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNFKSNFDLAEDAKLQ
LSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILT
FRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIV
KKTEVQTGGFSKESIRPKRNSDKLIARKKDWDPKKYGGFLWPTVAYSVLVVAKVEKGK
SKKLKSVKELLGITIMERSSFEKNPIDFLEAKGYKEVKKDLIIKLPKYSLFELENGRKRML
ASAKQLQKGNELALPSKYVNFLYLASHYEKLKGSPEDNEQKQLFVEQHKHYLDEIIEQIS
EFSKRVILADANLDKVLSAYNKHRDKPIREQAENIIHLFTLTRLGAPRAFKYFDTTIDPKQ
YRSTKEVLDATLIHQSITGLYETRIDLSQLGGDSGGSGGSGGSTNLSDIIEKETGKQLVIQE
SILMLPEEVEEVIGNKPESDILVHTAYDESTDENVMLLTSDAPEYKPWALVIQDSNGENK
IKMLSGGSGGSGGSTNLSDIIEKETGKQLVIQESILMLPEEVEEVIGNKPESDILVHTAYDE
STDENVMLLTSDAPEYKPWALVIQDSNGENKIKMLSGGSKRTADGSEFEPKKKRKVGS
GSKRPAATKKAGQAKKKKLE
SEQ ID NO: 7-Amino acid sequence of the SpRY-K918N-ABE8e-V106W 3xNLS adenine base editor:
MKRTADGSEFESPKKKRKVSEVEFSHEYWMRHALTLAKRARDEREVPVGAVLVLNNR
VIGEGWNRAIGLHDPTAHAEIMALRQGGLVMQNYRLIDATLYVTFEPCVMCAGAMIHS
RIGRVVFGWRNSKRGAAGSLMNVLNYPGMNHRVEITEGILADECAALLCDFYRMPRQV
FNAQKKAQSSINSGGSSGGSSGSETPGTSESATPESSGGSSGGSDKKYSIGLAIGTNSVGW
AVITDEYKVPSKKFKVLGNTDRHSIKKNLIGALLFDSGETAERTRLKRTARRRYTRRKN
RICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLSA
SMIKRYDEHHQDLTLLKALVRQQLPEKYKEIFFDQSKNGYAGYIDGGASQEEFYKFIKPI
LEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSID
NKVLTRSDKNRGKSDNVPSEEVVKKMKNYWRQLLNAKLITQRKFDNLTKAERGGLSE
LDKAGFINRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGRDFATVRKVLS
MPQVNIVKKTEVQTGGFSKESIRPKRNSDKLIARKKDWDPKKYGGFLWPTVAYSVLVV
AKVEKGKSKKLKSVKELLGITIMERSSFEKNPIDFLEAKGYKEVKKDLIIKLPKYSLFELE
NGRKRMLASAKQLQKGNELALPSKYVNFLYLASHYEKLKGSPEDNEQKQLFVEQHKH
YLDEIIEQISEFSKRVILADANLDKVLSAYNKHRDKPIREQAENIIHLFTLTRLGAPRAFKY
FDTTIDPKQYRSTKEVLDATLIHQSITGLYETRIDLSQLGGDSGGSKRTADGSEFEPKKKR
KVGSGSKRPAATKKAGQAKKKKLE
SEQ ID NO: 8-Nucleotide sequence of the SpRY-ABE8e-V106W 3xNLS adenine base editor:
atgaaacggacagccgacggaagcgagttcgagtcaccaaagaagaagcggaaagtctctgaggtggagttttcccacgagtactggat
gagacatgccctgaccctggccaagagggcacgggatgagagggaggtgcctgtgggagccgtgctggtgctgaacaatagagtgatc
ggcgagggctggaacagagccatcggcctgcacgacccaacagcccatgccgaaattatggccctgagacagggcggcctggtcatgc
agaactacagactgattgacgccaccctgtacgtgacattcgagccttgcgtgatgtgcgccggcgccatgatccactctaggatcggccg
cgtggtgtttggatggagaaattctaaaagaggcgccgcaggctccctgatgaacgtgctgaactaccccggcatgaatcaccgcgtcgaa
attaccgagggaatcctggcagatgaatgtgccgccctgctgtgcgatttctatcggatgcctagacaggtgttcaatgctcagaagaaggc
ccagagctccatcaactccggaggatctagcggaggctcctctggctctgagacacctggcacaagcgagagcgcaacacctgaaagca
gcgggggcagcagcggggggtcagacaagaagtacagcatcggcctggccatcggcaccaactctgtgggctgggccgtgatcaccg
acgagtacaaggtgcccagcaagaaattcaaggtgctgggcaacaccgaccggcacagcatcaagaagaacctgatcggagccctgct
gttcgacagcggcgaaacagccgagAGAacccggctgaagagaaccgccagaagaagatacaccagacggaagaaccggatctgc
tatctgcaagagatcttcagcaacgagatggccaaggtggacgacagcttcttccacagactggaagagtccttcctggtggaagaggata
agaagcacgagcggcaccccatcttcggcaacatcgtggacgaggtggcctaccacgagaagtaccccaccatctaccacctgagaaag
aaactggtggacagcaccgacaaggccgacctgcggctgatctatctggccctggcccacatgatcaagttccggggccacttcctgatcg
agggcgacctgaaccccgacaacagcgacgtggacaagctgttcatccagctggtgcagacctacaaccagctgttcgaggaaaacccc
atcaacgccagcggcgtggacgccaaggccatcctgtctgccagactgagcaagagcagacggctggaaaatctgatcgcccagctgcc
cggcgagaagaagaatggcctgttcggaaacctgattgccctgagcctgggcctgacccccaacttcaagagcaacttcgacctggccga
ggatgccaaactgcagctgagcaaggacacctacgacgacgacctggacaacctgctggcccagatcggcgaccagtacgccgacctg
tttctggccgccaagaacctgtccgacgccatcctgctgagcgacatcctgagagtgaacaccgagatcaccaaggcccccctgagcgcc
tctatgatcaagagatacgacgagcaccaccaggacctgaccctgctgaaagctctcgtgcggcagcagctgcctgagaagtacaaagag
attttcttcgaccagagcaagaacggctacgccggctacattgacggcggagccagccaggaagagttctacaagttcatcaagcccatcc
tggaaaagatggacggcaccgaggaactgctcgtgaagctgaacagagaggacctgctgcggaagcagcggaccttcgacaacggca
gcatcccccaccagatccacctgggagagctgcacgccattctgcggcggcaggaagatttttacccattcctgaaggacaaccgggaaa
agatcgagaagatcctgaccttccgcatcccctactacgtgggccctctggccaggggaaacagcagattcgcctggatgaccagaaaga
gcgaggaaaccatcaccccctggaacttcgaggaagtggtggacaagggcgcttccgcccagagcttcatcgagcggatgaccaacttc
gataagaacctgcccaacgagaaggtgctgcccaagcacagcctgctgtacgagtacttcaccgtgtataacgagctgaccaaagtgaaat
acgtgaccgagggaatgagaaagcccgccttcctgagcggcgagcagaaaaaggccatcgtggacctgctgttcaagaccaaccggaa
agtgaccgtgaagcagctgaaagaggactacttcaagaaaatcgagtgcttcgactccgtggaaatctccggcgtggaagatcggttcaac
gcctccctgggcacataccacgatctgctgaaaattatcaaggacaaggacttcctggacaatgaggaaaacgaggacattctggaagata
tcgtgctgaccctgacactgtttgaggacagagagatgatcgaggaacggctgaaaacctatgcccacctgttcgacgacaaagtgatgaa
gcagctgaagcggcggagatacaccggctggggcaggctgagccggaagctgatcaacggcatccgggacaagcagtccggcaaga
caatcctggatttcctgaagtccgacggcttcgccaacagaaacttcatgcagctgatccacgacgacagcctgacctttaaagaggacatc
cagaaagcccaggtgtccggccagggcgatagcctgcacgagcacattgccaatctggccggcagccccgccattaagaagggcatcct
gcagacagtgaaggtggtggacgagctcgtgaaagtgatgggccggcacaagcccgagaacatcgtgatcgaaatggccagagagaa
ccagaccacccagaagggacagaagaacagccgcgagagaatgaagcggatcgaagagggcatcaaagagctgggcagccagatcc
tgaaagaacaccccgtggaaaacacccagctgcagaacgagaagctgtacctgtactacctgcagaatggggggatatgtacgtggac
caggaactggacatcaaccggctgtccgactacgatgtggaccatatcgtgcctcagagctttctgaaggacgactccatcgacaacaagg
tgctgaccagaagcgacaagaaccggggcaagagcgacaacgtgccctccgaagaggtcgtgaagaagatgaagaactactggcggc
agctgctgaacgccaagctgattacccagagaaagttcgacaatctgaccaaggccgagagaggcggcctgagcgaactggataaggc
cggcttcatcaagagacagctggtggaaacccggcagatcacaaagcacgtggcacagatcctggactcccggatgaacactaagtacg
acgagaatgacaagctgatccgggaagtgaaagtgatcaccctgaagtccaagctggtgtccgatttccggaaggatttccagttttacaaa
gtgcgcgagatcaacaactaccaccacgcccacgacgcctacctgaacgccgtcgtgggaaccgccctgatcaaaaagtaccctaagct
ggaaagcgagttcgtgtacggcgactacaaggtgtacgacgtgcggaagatgatcgccaagagcgagcaggaaatcggcaaggctacc
gccaagtacttcttctacagcaacatcatgaactttttcaagaccgagattaccctggccaacggcgagatccggaagcggcctctgatcga
gacaaacggcgaaaccggggagatcgtgtgggataagggccgggattttgccaccgtgcggaaagtgctgagcatgccccaagtgaata
tcgtgaaaaagaccgaggtgcagacaggcggcttcagcaaagagtctatcAGAcccaagaggaacagcgataagctgatcgccagaa
agaaggactgggaccctaagaagtacggcggcttcCTTTGGcccaccgtggcctattctgtgctggtggtggccaaagtggaaaagg
gcaagtccaagaaactgaagagtgtgaaagagctgctggggatcaccatcatggaaagaagcagcttcgagaagaatcccatcgactttct
ggaagccaagggctacaaagaagtgaaaaaggacctgatcatcaagctgcctaagtactccctgttcgagctggaaaacggccggaaga
gaatgctggcctctgccAAGCaactgcagaagggaaacgaactggccctgccctccaaatatgtgaacttcctgtacctggccagcca
ctatgagaagctgaagggctcccccgaggataatgagcagaaacagctgtttgtggaacagcacaagcactacctggacgagatcatcga
gcagatcagcgagttctccaagagagtgatcctggccgacgctaatctggacaaagtgctgtccgcctacaacaagcaccgggataagcc
catcagagagcaggccgagaatatcatccacctgtttaccctgaccaGActgggagcccctAGAgccttcaagtactttgacaccacca
tcgaccCTaagCAAtacaGAagcaccaaagaggtgctggacgccaccctgatccaccagagcatcaccggcctgtacgagacac
ggatcgacctgtctcagctgggaggtgactctggcggctcaaaaagaaccgccgacggcagcgaattcgagcccaagaagaagaggaa
agtcggcagcggaagcaaaaggccggcggccacgaaaaaggccggccaggcaaaaaagaaaaagctcgagtaa
SEQ ID NO: 9-Nucleotide sequence of the SpRY-evoAPOBEC1-BE4 3xNLS adenine base editor:
atgaaacggacagccgacggaagcgagttcgagtcaccaaagaagaagcggaaagtcagttcaaagactgggcctgtcgccgtcgatc
caaccctgcgccgccggattgaacctcacgagtttgaagtgttctttgacccccgggagctgagaaaggagacatgcctgctgtacgagat
caactggggaggcaggcactccatctggaggcacacctctcagaacacaaataagcacgtggaggtgaacttcatcgagaagtttaccac
agagcggtacttctgccccaataccagatgtagcatcacatggtttctgagctggtccccttgcggagagtgtagcagggccatcaccgagt
tcctgtccagatatccaaatgtgacactgtttatctacatcgccaggctgtatcacctggcaaacccaaggaataggcagggcctgcgcgatc
tgatcagctccggcgtgaccatccagatcatgacagagcaggagtccggctactgctggcacaacttcgtgaattattctcctagcaacgag
tcccactggcctaggtacccacacctgtgggtgcgcctgtacgtgctggagctgtattgcatcatcctgggcctgcccccttgtctgaatat
cctgcggagaaagcagagccagctgacctcctttacaatcgccctgcagtcttgtcactatcagaggctgccaccccacatcctgtgggccac
aggcctgaagtctggcggatctagcggaggatcctctggcagcgagacaccaggaacaagcgagtcagcaacaccagagagcagtgg
cggcagcagcggcggcagcgacaagaagtacagcatcggcctggccatcggcaccaactctgtgggctgggccgtgatcaccgacga
gtacaaggtgcccagcaagaaattcaaggtgctgggcaacaccgaccggcacagcatcaagaagaacctgatcggagccctgctgttcg
acagcggcgaaacagccgagAGAacccggctgaagagaaccgccagaagaagatacaccagacggaagaaccggatctgctatct
gcaagagatcttcagcaacgagatggccaaggtggacgacagcttcttccacagactggaagagtccttcctggtggaagaggataagaa
gcacgagcggcaccccatcttcggcaacatcgtggacgaggtggcctaccacgagaagtaccccaccatctaccacctgagaaagaaac
tggtggacagcaccgacaaggccgacctgcggctgatctatctggccctggcccacatgatcaagttccggggccacttcctgatcgagg
gcgacctgaaccccgacaacagcgacgtggacaagctgttcatccagctggtgcagacctacaaccagctgttcgaggaaaaccccatca
acgccagcggcgtggacgccaaggccatcctgtctgccagactgagcaagagcagacggctggaaaatctgatcgcccagctgcccgg
cgagaagaagaatggcctgttcggaaacctgattgccctgagcctgggcctgacccccaacttcaagagcaacttcgacctggccgagga
tgccaaactgcagctgagcaaggacacctacgacgacgacctggacaacctgctggcccagatcggcgaccagtacgccgacctgtttct
ggccgccaagaacctgtccgacgccatcctgctgagcgacatcctgagagtgaacaccgagatcaccaaggcccccctgagcgcctcta
tgatcaagagatacgacgagcaccaccaggacctgaccctgctgaaagctctcgtgcggcagcagctgcctgagaagtacaaagagattt
tcttcgaccagagcaagaacggctacgccggctacattgacggcggagccagccaggaagagttctacaagttcatcaagcccatcctgg
aaaagatggacggcaccgaggaactgctcgtgaagctgaacagagaggacctgctgcggaagcagcggaccttcgacaacggcagca
tcccccaccagatccacctgggagagctgcacgccattctgcggcggcaggaagatttttacccattcctgaaggacaaccgggaaaaga
tcgagaagatcctgaccttccgcatcccctactacgtgggccctctggccaggggaaacagcagattcgcctggatgaccagaaagagcg
aggaaaccatcaccccctggaacttcgaggaagtggtggacaagggcgcttccgcccagagcttcatcgagcggatgaccaacttcgata
agaacctgcccaacgagaaggtgctgcccaagcacagcctgctgtacgagtacttcaccgtgtataacgagctgaccaaagtgaaatacgt
gaccgagggaatgagaaagcccgccttcctgagcggcgagcagaaaaaggccatcgtggacctgctgttcaagaccaaccggaaagtg
accgtgaagcagctgaaagaggactacttcaagaaaatcgagtgcttcgactccgtggaaatctccggcgtggaagatcggttcaacgcct
ccctgggcacataccacgatctgctgaaaattatcaaggacaaggacttcctggacaatgaggaaaacgaggacattctggaagatatcgt
gctgaccctgacactgtttgaggacagagagatgatcgaggaacggctgaaaacctatgcccacctgttcgacgacaaagtgatgaagca
gctgaagcggcggagatacaccggctggggcaggctgagccggaagctgatcaacggcatccgggacaagcagtccggcaagacaat
cctggatttcctgaagtccgacggcttcgccaacagaaacttcatgcagctgatccacgacgacagcctgacctttaaagaggacatccaga
aagcccaggtgtccggccagggcgatagcctgcacgagcacattgccaatctggccggcagccccgccattaagaagggcatcctgca
gacagtgaaggtggtggacgagctcgtgaaagtgatgggccggcacaagcccgagaacatcgtgatcgaaatggccagagagaacca
gaccacccagaagggacagaagaacagccgcgagagaatgaagcggatcgaagagggcatcaaagagctgggcagccagatcctga
aagaacaccccgtggaaaacacccagctgcagaacgagaagctgtacctgtactacctgcagaatggggggatatgtacgtggaccag
gaactggacatcaaccggctgtccgactacgatgtggaccatatcgtgcctcagagctttctgaaggacgactccatcgacaacaaggtgct
gaccagaagcgacaagaaccggggcaagagcgacaacgtgccctccgaagaggtcgtgaagaagatgaagaactactggcggcagct
gctgaacgccaagctgattacccagagaaagttcgacaatctgaccaaggccgagagaggcggcctgagcgaactggataaggccggc
ttcatcaagagacagctggtggaaacccggcagatcacaaagcacgtggcacagatcctggactcccggatgaacactaagtacgacga
gaatgacaagctgatccgggaagtgaaagtgatcaccctgaagtccaagctggtgtccgatttccggaaggatttccagttttacaaagtgc
gcgagatcaacaactaccaccacgcccacgacgcctacctgaacgccgtcgtgggaaccgccctgatcaaaaagtaccctaagctggaa
agcgagttcgtgtacggcgactacaaggtgtacgacgtgcggaagatgatcgccaagagcgagcaggaaatcggcaaggctaccgcca
agtacttcttctacagcaacatcatgaactttttcaagaccgagattaccctggccaacggcgagatccggaagcggcctctgatcgagaca
aacggcgaaaccggggagatcgtgtgggataagggccgggattttgccaccgtgcggaaagtgctgagcatgccccaagtgaatatcgt
gaaaaagaccgaggtgcagacaggcggcttcagcaaagagtctatcA Ggcccaagaggaacagcgataagctgatcgccagaaaga
aggactgggaccctaagaagtacggcggcttcCTGTGGcccaccgtggcctattctgtgctggtggtggccaaagtggaaaagggc
aagtccaagaaactgaagagtgtgaaagagctgctggggatcaccatcatggaaagaagcagcttcgagaagaatcccatcgactttctgg
aagccaagggctacaaagaagtgaaaaaggacctgatcatcaagctgcctaagtactccctgttcgagctggaaaacggccggaagaga
atgctggcctctgccAAGCAGctgcagaagggaaacgaactggccctgccctccaaatatgtgaacttcctgtacctggccagccact
atgagaagctgaagggctcccccgaggataatgagcagaaacagctgtttgtggaacagcacaagcactacctggacgagatcatcgag
cagatcagcgagttctccaagagagtgatcctggccgacgctaatctggacaaagtgctgtccgcctacaacaagcaccgggataagccc
atcagagagcaggccgagaatatcatccacctgtttaccctgaccaGGctgggagcccctAGAgccttcaagtactttgacaccaccat
cgaccCCaagCAgtacaGGagcaccaaagaggtgctggacgccaccctgatccaccagagcatcaccggcctgtacgagacacg
gatcgacctgtctcagctgggaggtgacagcggcgggagcggcgggagcggggggagcactaatctgagcgacatcattgagaagga
gactgggaaacagctggtcattcaggagtccatcctgatgctgcctgaggaggtggaggaagtgatcggcaacaagccagagtctgacat
cctggtgcacaccgcctacgacgagtccacagatgagaatgtgatgctgctgacctctgacgcccccgagtataagccttgggccctggtc
atccaggattctaacggcgagaataagatcaagatgctgagcggaggatccggaggatctggaggcagcaccaacctgtctgacatcatc
gagaaggagacaggcaagcagctggtcatccaggagagcatcctgatgctgcccgaagaagtcgaagaagtgatcggaaacaagcctg
agagcgatatcctggtccataccgcctacgacgagagtaccgacgaaaatgtgatgctgctgacatccgacgccccagagtataagccct
gggctctggtcatccaggattccaacggagagaacaaaatcaaaatgtgtctggcggctcaaaaagaaccgccgacggcagcgaattc
gagcccaagaagaagaggaaagtcggcagcggaagcaaaaggccggcggccacgaaaaaggccggccaggcaaaaaagaaaaag
ctcgagtaa
SEQ ID NO: 10-Nucleotide sequence of the SpRY-K918N-ABE8e-V106W 3xNLS adenine base editor:
atgaaacggacagccgacggaagcgagttcgagtcaccaaagaagaagcggaaagtctctgaggtggagttttcccacgagtactggat
gagacatgccctgaccctggccaagagggcacgggatgagagggaggtgcctgtgggagccgtgctggtgctgaacaatagagtgatc
ggcgagggctggaacagagccatcggcctgcacgacccaacagcccatgccgaaattatggccctgagacagggggcctggtcatgc
agaactacagactgattgacgccaccctgtacgtgacattcgagccttgcgtgatgtgcgccggcgccatgatccactctaggatcggccg
cgtggtgtttggatggagaaattctaaaagaggcgccgcaggctccctgatgaacgtgctgaactaccccggcatgaatcaccgcgtcgaa
attaccgagggaatcctggcagatgaatgtgccgccctgctgtgcgatttctatcggatgcctagacaggtgttcaatgctcagaagaaggc
ccagagctccatcaactccggaggatctagcggaggctcctctggctctgagacacctggcacaagcgagagcgcaacacctgaaagca
gcgggggcagcagcggggggtcagacaagaagtacagcatcggcctggccatcggcaccaactctgtgggctgggccgtgatcaccg
acgagtacaaggtgcccagcaagaaattcaaggtgctgggcaacaccgaccggcacagcatcaagaagaacctgatcggagccctgct
gttcgacagcggcgaaacagccgagAGAacccggctgaagagaaccgccagaagaagatacaccagacggaagaaccggatctgc
tatctgcaagagatcttcagcaacgagatggccaaggtggacgacagcttcttccacagactggaagagtccttcctggtggaagaggata
agaagcacgagcggcaccccatcttcggcaacatcgtggacgaggtggcctaccacgagaagtaccccaccatctaccacctgagaaag
aaactggtggacagcaccgacaaggccgacctgcggctgatctatctggccctggcccacatgatcaagttccggggccacttcctgatcg
agggcgacctgaaccccgacaacagcgacgtggacaagctgttcatccagctggtgcagacctacaaccagctgttcgaggaaaacccc
atcaacgccagcggcgtggacgccaaggccatcctgtctgccagactgagcaagagcagacggctggaaaatctgatcgcccagctgcc
cggcgagaagaagaatggcctgttcggaaacctgattgccctgagcctgggcctgacccccaacttcaagagcaacttcgacctggccga
ggatgccaaactgcagctgagcaaggacacctacgacgacgacctggacaacctgctggcccagatcggcgaccagtacgccgacctg
tttctggccgccaagaacctgtccgacgccatcctgctgagcgacatcctgagagtgaacaccgagatcaccaaggcccccctgagcgcc
tctatgatcaagagatacgacgagcaccaccaggacctgaccctgctgaaagctctcgtgcggcagcagctgcctgagaagtacaaagag
attttcttcgaccagagcaagaacggctacgccggctacattgacggcggagccagccaggaagagttctacaagttcatcaagcccatcc
tggaaaagatggacggcaccgaggaactgctcgtgaagctgaacagagaggacctgctgcggaagcagcggaccttcgacaacggca
gcatcccccaccagatccacctgggagagctgcacgccattctgcggcggcaggaagatttttacccattcctgaaggacaaccgggaaa
agatcgagaagatcctgaccttccgcatcccctactacgtgggccctctggccaggggaaacagcagattcgcctggatgaccagaaaga
gcgaggaaaccatcaccccctggaacttcgaggaagtggtggacaagggcgcttccgcccagagcttcatcgagcggatgaccaacttc
gataagaacctgcccaacgagaaggtgctgcccaagcacagcctgctgtacgagtacttcaccgtgtataacgagctgaccaaagtgaaat
acgtgaccgagggaatgagaaagcccgccttcctgagcggcgagcagaaaaaggccatcgtggacctgctgttcaagaccaaccggaa
agtgaccgtgaagcagctgaaagaggactacttcaagaaaatcgagtgcttcgactccgtggaaatctccggcgtggaagatcggttcaac
gcctccctgggcacataccacgatctgctgaaaattatcaaggacaaggacttcctggacaatgaggaaaacgaggacattctggaagata
tcgtgctgaccctgacactgtttgaggacagagagatgatcgaggaacggctgaaaacctatgcccacctgttcgacgacaaagtgatgaa
gcagctgaagcggggagatacaccggctggggcaggctgagccggaagctgatcaacggcatccgggacaagcagtccggcaaga
caatcctggatttcctgaagtccgacggcttcgccaacagaaacttcatgcagctgatccacgacgacagcctgacctttaaagaggacatc
cagaaagcccaggtgtccggccaggggatagcctgcacgagcacattgccaatctggccggcagccccgccattaagaagggcatcct
gcagacagtgaaggtggtggacgagctcgtgaaagtgatgggccggcacaagcccgagaacatcgtgatcgaaatggccagagagaa
ccagaccacccagaagggacagaagaacagccgcgagagaatgaagcggatcgaagagggcatcaaagagctgggcagccagatcc
tgaaagaacaccccgtggaaaacacccagctgcagaacgagaagctgtacctgtactacctgcagaatggggggatatgtacgtggac
caggaactggacatcaaccggctgtccgactacgatgtggaccatatcgtgcctcagagctttctgaaggacgactccatcgacaacaagg
tgctgaccagaagcgacaagaaccggggcaagagcgacaacgtgccctccgaagaggtcgtgaagaagatgaagaactactggcggc
agctgctgaacgccaagctgattacccagagaaagttcgacaatctgaccaaggccgagagaggcggcctgagcgaactggataaggc
cggcttcatcaaCagacagctggtggaaacccggcagatcacaaagcacgtggcacagatcctggactcccggatgaacactaagtacg
acgagaatgacaagctgatccgggaagtgaaagtgatcaccctgaagtccaagctggtgtccgatttccggaaggatttccagttttacaaa
gtgcgcgagatcaacaactaccaccacgcccacgacgcctacctgaacgccgtcgtgggaaccgccctgatcaaaaagtaccctaagct
ggaaagcgagttcgtgtacggcgactacaaggtgtacgacgtgcggaagatgatcgccaagagcgagcaggaaatcggcaaggctacc
gccaagtacttcttctacagcaacatcatgaactttttcaagaccgagattaccctggccaacggcgagatccggaagcggcctctgatcga
gacaaacggcgaaaccggggagatcgtgtgggataagggccgggattttgccaccgtgcggaaagtgctgagcatgccccaagtgaata
tcgtgaaaaagaccgaggtgcagacaggcggcttcagcaaagagtctatcAGAcccaagaggaacagcgataagctgatcgccagaa
agaaggactgggaccctaagaagtacggcggcttcCTTTGGcccaccgtggcctattctgtgctggtggtggccaaagtggaaaagg
gcaagtccaagaaactgaagagtgtgaaagagctgctggggatcaccatcatggaaagaagcagcttcgagaagaatcccatcgactttct
ggaagccaagggctacaaagaagtgaaaaaggacctgatcatcaagctgcctaagtactccctgttcgagctggaaaacggccggaaga
gaatgctggcctctgccAAGCaactgcagaagggaaacgaactggccctgccctccaaatatgtgaacttcctgtacctggccagcca
ctatgagaagctgaagggctcccccgaggataatgagcagaaacagctgtttgtggaacagcacaagcactacctggacgagatcatcga
gcagatcagcgagttctccaagagagtgatcctggccgacgctaatctggacaaagtgctgtccgcctacaacaagcaccgggataagcc
catcagagagcaggccgagaatatcatccacctgtttaccctgaccaGActgggagcccctAGAgccttcaagtactttgacaccacca
tcgaccCTaagCAAtacaGAagcaccaaagaggtgctggacgccaccctgatccaccagagcatcaccggcctgtacgagacac
ggatcgacctgtctcagctgggaggtgactctggcggctcaaaaagaaccgccgacggcagcgaattcgagcccaagaagaagaggaa
agtcggcagcggaagcaaaaggccggcggccacgaaaaaggccggccaggcaaaaaagaaaaagctcgag

### CRISPR Guide RNAs

The terms “gRNA,” “guide RNA,” and “CRISPR guide sequence” are used interchangeably throughout and refer to a nucleic acid comprising a sequence that determines the specificity of a Cas DNA binding protein of a CRISPR/Cas system. A gRNA hybridizes to (e.g., is complementary to, either partially or completely) a target nucleic acid sequence in the genome of a host cell and promotes the specific association or targeting of an RNA-guided nuclease, such as a Cas9 or a Cpf1, to a target sequence. gRNAs can be unimolecular (comprising a single RNA molecule, and referred to alternatively as chimeric or sgRNAs), or modular (comprising more than one, and typically two, separate RNA molecules, such as a CRISPR RNA (crRNA) and a trans-activating CRISPR RNA (tracrRNA), which are usually associated with one another, for instance by duplexing or hybridizing). Thus, in some instances, the gRNA refers collectively to the crRNA and the tracrRNA (for instance, when a Cas9 nuclease is being used—in those instances, the guide RNA may be referred to as a single guide RNA, i.e., sgRNA). In other instances, the gRNA refers only to the crRNA (for instance, when a Cpf1 endonuclease is being used).

Guide RNAs, whether unimolecular or modular, include a “targeting domain” that is fully or partially complementary to a target domain within a target sequence. Targeting domains are referred to by various names in the literature, including without limitation “guide sequences,” “complementarity regions,” “spacers,” and generically as “crRNAs.” The gRNA or portion thereof that hybridizes to the target nucleic acid can include 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is 10-30, or 15-25, nucleotides in length. In some embodiments, the gRNA sequence has at least 50%, at least 55%, at least 60%, at least 65%, at least 70%, at least 75%, at least 80%, at least 85%, at least 90%, at least 95%, at least 96%, at least 97%, at least 98%, at least 99%, or 100% complementary to a target nucleic acid.

In addition to the targeting domains, gRNAs typically (but not necessarily) include a plurality of domains that may influence the formation or activity of Cas9/gRNA complexes. This includes, for example, one or more polyA tracts, which can be recognized by RNA polymerases as a termination signal, and two or more additional duplexed regions that are involved in nuclease activity in vivo but not necessarily in vitro. While this description is focused on gRNAs for use with Cas9, other RNA-guided nucleases exist that utilize gRNAs that differ in some ways from those described herein. The design of other gRNAs is further described, for example, in International Publication No. WO 2019/084168.

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

Exemplary guide crRNAs for editing CD38 genes are provided in Table 1 below. As is well known, selection of gRNA sequences can depend on factors such as the number of predicted on-target and/or off-target binding sites. In some embodiments, the gRNA sequence is selected to maximize potential on-target and minimize potential off-target sites.

In some embodiments, multiple gRNAs are introduced into the cell. In some embodiments, the two or more guide RNAs are transfected into cells in equimolar amounts. In some embodiments, the two or more guide RNAs are provided in amounts that are not equimolar. In some embodiments, the two or more guide RNAs are provided in amounts that are optimized so that editing of each target occurs at equal frequency. In some embodiments, the two or more guide RNAs are provided in amounts that are optimized so that editing of each target occurs at optimal frequency.

Provided herein is a polynucleotide, suitable for use as a guide spacer sequence, having a sequence that is at least 75%, at least 80%, at least 85%, at least 90%, at least 95%, at least 96%, at least 97%, at least 98%, at least 99%, or 100% identical to the sequences set forth in Table 1 (SEQ ID NOS: 11-13). Such polynucleotides are suitable for use as the crRNA segment in a guide RNA that forms genetically modified CD38 genes that results in a polypeptide bearing a mutation at position S274, respectively.

TABLE 1

| | | SEQ | | | Base | | |
|---|---|---|---|---|---|---|---|
| Target | gRNA | ID | Guide spacer | | Editor | PAM | Cas |
| Gene | name | NO: | Sequence (DNA) | Mutation | Type | Sequence | Requirement |
| CD38 | CD38_ | 11 | TTTCCTGCAA | S274F | Cytosine | AGG | Cas9 |
| | gRNA_ | | GAATATCTAC | | | | |
| | 1 | | | | | | |
| CD38 | CD38_ | 12 | TTTTCCTGCA | S274F | Cytosine | CAG | SpRY-Cas9 |
| | gRNA_ | | AGAATATCTA | | | | |
| | 2 | | | | | | |
| CD38 | CD38_ | 13 | ATTTTCCTGC | S274F | Cytosine | ACA | SpRY-Cas9 |
| | gRNA_ | | AAGAATATCT | | | | |
| | 3 | | | | | | |

vi. Genetically Engineered Cells (e.g., HSPCs and/or T cells)

Provided herein are “genetically engineered cells.” This refers to a cell that includes a polynucleotide that the cell does not naturally possess. Also provided herein are methods of producing the genetically engineered cells (e.g., HSPCs and/or T cells) as described herein, which include edited genes for expressing one or more cell-surface antigens in mutated form.

Methods of producing genetically engineered cells can involve providing a cell and introducing into the cell components of a nucleotide-guided gene editing system for genome editing. In some embodiments, a nucleic acid that comprises a gRNA that hybridizes or is predicted to hybridize to a portion of the nucleotide sequence that encodes the cell-surface antigen is introduced into the cell. In some embodiments, the gRNA is introduced into the cell on a vector. In some embodiments, a Cas endonuclease is introduced into the cell. In some embodiments, the Cas endonuclease is introduced into the cell as a nucleic acid encoding a Cas endonuclease. In some embodiments, the gRNA and a nucleotide sequence encoding a Cas endonuclease are introduced into the cell on the same nucleic acid (e.g., the same vector). In some embodiments, the Cas endonuclease is introduced into the cell in the form of a protein. In some embodiments, the Cas endonuclease and the gRNA are pre-formed in vitro and are introduced to the cell in as a ribonucleoprotein complex.

### Genetically Engineered Cells Expressing Mutant CD38

In some embodiments, the cell-surface protein is CD38. The amino acid sequence of wild-type CD38 is known (uniprot.org/uniprotkb/P28907/entry) (Accession No. AAA68482.1).

In some embodiments, the methods described herein involve genetically engineering a population of cells (e.g., HSPCs or T cells) using a nucleotide-guided gene editing system, such as a Cas nuclease (or variant thereof). In some embodiments, the methods described herein involve genetically engineering a gene encoding a cell-surface antigen in a population of cells (e.g., HSPCs or T cells) using a Cas nuclease or variant thereof (e.g., SpCas9 or AsCpf1). In some embodiments, the methods described herein involve genetically modifying or editing a CD38 gene in the population of cells (e.g., HSPCs or T cells) using the Cas nuclease. In some embodiments, the methods described herein involve genetically engineering CD38 by mutating position S274 in the amino acid sequence of CD38 in a population of HSPCs or T cells using a nucleotide-guided gene editing system. In some embodiments, the methods described herein involve genetically engineering a mutant CD38 gene in a population of HSPCs or T cells using a nucleotide-guided gene editing system, including a guide sequence provided by any one of SEQ ID NOs: 11-13.

In some embodiments, the genetically engineered HSPC or T cell includes a genetically engineered CD38 gene, wherein the genetically engineered CD38 gene encodes a protein that has reduced binding to a therapeutic anti-CD38 antibody (e.g., daratumumab). In some embodiments, the genetically engineered CD38 gene encodes a protein that has a mutation at position S274 of CD38. In some instances, the mutation at position S274 is S274F. The amino acid sequence of this genetically engineered CD38 is provided below:

SEQ ID NO: 52 (CD38 polypeptide with mutation at S274F):
MANCEFSPVSGDKPCCRLSRRAQLCLGVSILVLILVVVLAVVVPRWRQQWSGPGTTKRF
PETVLARCVKYTEIHPEMRHVDCQSVWDAFKGAFISKHPCNITEEDYQPLMKLGTQTVP
CNKILLWSRIKDLAHQFTQVQRDMFTLEDTLLGYLADDLTWCGEFNTSKINYQSCPDW
RKDCSNNPVSVFWKTVSRRFAEAACDVVHVMLNGSRSKIFDKNSTFGSVEVHNLQPEK
VQTLEAWVIHGGREDSRDLCQDPTIKELESIISKRNIQFFCKNIYRPDKFLQCVKNPEDSS
CTSEI

In some embodiments, provided herein is a polypeptide sequence that is at least 80%, at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 52, wherein the polypeptide sequence comprises a mutation at S274F and wherein the polypeptide sequence has reduced binding to a therapeutic anti-CD38 antibody (e.g., daratumumab). Also provided herein are nucleic acids encoding the polypeptide sequence, a vector comprising the nucleic acid, a cell comprising the nucleic acid or the vector, and a method of making a polypeptide, the method comprising culturing the cell under conditions that allow for the expression of the polypeptide and optionally isolating the polypeptide.

### Genetically Engineered Cells (e.g., HSPCs) Expressing a Multiplex System

In some embodiments, the cell-surface proteins CD38 may be combined with other genetic engineering strategies, such as: i) other epitope editing on other target proteins; ii) other therapeutic base or prime editing approaches (e.g., BCL11A erythroid enhancer); and iii) conventional gene therapy with integrating vectors. For example, in some embodiments, this can be accomplished by transfecting two or more guide RNAs for different target surface proteins concurrently with each other. In some embodiments, the two or more guide RNAs are provided sequentially or consecutively, i.e., in two or more separate transfections.

### II. Immunotherapy Agents Specific to Cell-Surface Antigens

Cytotoxic agents targeting cells (e.g., cancer cells) expressing a cell-surface antigen can be co-used with the genetically engineered cells (e.g., HSPCs or T cells) as described herein. As used herein, the term “cytotoxic agent” refers to any agent that can directly or indirectly induce cytotoxicity of a target cell, which expresses the specific cell-surface antigen (e.g., a target cancer cell). Such a cytotoxic agent can comprise a protein-binding fragment that binds and targets an epitope of the specific cell-surface antigen.

i. Therapeutic Antibodies/Antibody-Drug Conjugates

Herein, a genetically engineered gene is engineered such that its encoded protein has reduced binding to a therapeutic antibody. In this context, a “therapeutic” antibody refers to an antibody that ameliorates one or more existing symptoms or clinical signs associated with a condition, such as a hematological condition. An “antibody” refers to a molecule that contains at least one antigen binding site that immunospecifically binds to a particular antigen target of interest. The term “antibody” thus includes, but is not limited to, a full length antibody and/or its variants, a fragment thereof, peptibodies, and variants thereof, monoclonal antibodies (including full-length monoclonal antibodies), polyclonal antibodies, multispecific antibodies (e.g., bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Thus, as used herein, the term “antibody” encompasses antibody fragments capable of binding to a biological molecule (such as an antigen or receptor) or a portion thereof, including but not limited to Fab, Fab′ and F(ab′)2, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments.

In some embodiments, a cytotoxic agent includes a therapeutic antibody, which can be conjugated to a drug (e.g., an anti-cancer drug) to form an antibody-drug conjugate (ADC). In some embodiments, the agent is an antibody-drug conjugate. In some embodiments, the antibody-drug conjugate comprises an epitope binding fragment and a toxin or drug that induces cytotoxicity in a target cell.

In some embodiments, the therapeutic anti-CD38 antibody is daratumumab.

Toxins or drugs compatible for use in antibody-drug conjugates are well known in the art and will be evident to one of ordinary skill in the art. See, e.g., Peters et al. Biosci. Rep. (2015) 35(4): e00225, Beck et al. Nature Reviews Drug Discovery (2017) 16:315-337; Marin-Acevedo et al. J. Hematol. Oncol. (2018) 11: 8; Elgundi et al. Advanced Drug Delivery Reviews (2017) 122: 2-19. In some embodiments, the antibody-drug conjugate can further comprise a linker (e.g., a peptide linker, such as a cleavable linker or a non-cleavable linker) attaching the antibody and drug molecule. Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1 A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/IILN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861.

In some embodiments, binding of the antibody-drug conjugate to the epitope of the cell-surface protein induces internalization of the antibody-drug conjugate, and the drug (or toxin) can be released intracellularly. In some embodiments, binding of the antibody-drug conjugate to the epitope of a cell-surface protein induces internalization of the toxin or drug, which allows the toxin or drug to kill the cells expressing the cell surface protein (target cells). In some embodiments, binding of the antibody-drug conjugate to the epitope of a cell-surface protein induces internalization of the toxin or drug, which can regulate the activity of the cell expressing the cell surface protein (target cells). The type of toxin or drug used in the antibody-drug conjugates described herein is not limited to any specific type.

In some embodiments, two or more (e.g., 2, 3, 4, 5 or more) epitopes of a cell-surface antigen have been modified, enabling two or more (e.g., 2, 3, 4, 5 or more) different cytotoxic agents (e.g., two ADCs) to be targeted to the two or more epitopes. In some embodiments, the toxins carried by the ADCs could work synergistically to enhance efficacy (e.g., death of the target cells). In some embodiments, epitopes of two or more (e.g., 2, 3, 4, 5 or more) cell-surface proteins have been modified, enabling two or more (e.g., 2, 3, 4, 5 or more) different cytotoxic agents (e.g., two ADCs) to be targeted to epitopes of the two or more cell-surface antigens. In some embodiments, one or more (e.g., 1, 2, 3, 4, 5 or more) epitopes of a cell-surface antigen have been modified and one or more (e.g., 1, 2, 3, 4, 5 or more) epitopes of an additional cell-surface protein have been modified, enabling two or more (e.g., 2, 3, 4, 5 or more) different cytotoxic agents (e.g., two ADCs) to be targeted to epitopes of the cell-surface antigen and epitopes of additional cell-surface antigen. In some embodiments, targeting of more than one cell-surface antigen or a cell-surface antigen and one or more additional cell-surface protein/antigen can reduce relapse of a hematopoietic malignancy.

In some embodiments, the methods described herein involve administering ADCs that target an epitope of a cell-surface antigen that is mutated in the population of genetically engineered hematopoietic cells. In some embodiments, the methods described herein involve administering ADCs that target an epitope of a cell-surface antigen that is mutated in the population of genetically engineered cells (e.g., HSPCs or T cells) and one or more additional cytotoxic agents that can target one or more additional cell-surface proteins. In some embodiments, the agents could work synergistically to enhance efficacy by targeting more than one cell-surface protein.

An ADC described herein can be used as a follow-on treatment to subjects who have undergone the combined therapy as described herein.

In some embodiments, the methods described herein involve administering to the subject a population of genetically engineered cells lacking a non-essential epitope in a cell-surface antigen (e.g., type 1 or type 2) and one or more immunotherapeutic agents (e.g., ADCs) that target cells expressing the cell-surface antigen. In any of the embodiments described herein, one or more additional immunotherapeutic agents can be further administered to the subject (e.g., targeting one or more additional epitopes and/or antigens), for example, if the hematopoietic malignancy relapses.

ii. Immune Cells Expressing Chimeric Antigen Receptors (CARs)

In some embodiments, the cytotoxic agent that targets an epitope of a specific cell-surface antigen as described herein is an immune cell that expresses a chimeric antigen receptor (CAR), which comprises an epitope binding fragment (e.g., a single-chain antibody) capable of binding to the epitope of the cell surface protein (e.g., CD38).

As used herein, a “chimeric antigen receptor” (CAR or simply chimeric receptor) refers to a non-naturally occurring molecule that can be expressed on the surface of a host cell and comprises binding domain that provides specificity of the CAR (e.g., an epitope binding fragment that binds to an epitope of a cell-surface lineage-specific protein). In general, CARs include at least two domains that are derived from different molecules.

Recognition of a target cell (e.g., a cancer cell) having the epitope of the specific protein on its cell surface by the epitope binding fragment of a CAR transduces an activation signal to the signaling domain(s) (e.g., co-stimulatory signaling domain and/or the cytoplasmic signaling domain) of the CAR, which can activate an effector function in the immune cell expressing the CAR.

In some embodiments, the immune cell expresses more than one CAR (e.g., 2, 3, 4, 5 or more), referred to as a bispecific or multi-specific immune cell. In some embodiments, the immune cell expresses more than one CAR, at least one of which targets an epitope of a cell-surface antigen. In some embodiments, the immune cell expresses more than one CAR, each of which targets an epitope of a specific cell-surface antigen. In some embodiments, the immune cell expresses more than one CAR, at least one of which targets an epitope of a cell-surface antigen and at least one of which targets an epitope of an additional cell-surface antigen. In some embodiments, targeting of more than one cell-surface protein or a cell-surface protein and one or more additional cell-surface protein can reduce relapse of a hematopoietic malignancy. In some embodiments, the immune cell expresses a CAR that targets more than one epitope (e.g., more than one epitope of one antigen or epitopes of more than one antigen), referred to as a bispecific CAR.

In some embodiments, epitopes of two or more lineage-specific cell-surface proteins are targeted by cytotoxic agents. In some embodiments, two or more CARs are expressed in the same immune cell, e.g., bispecific chimeric receptors. Such cells can be used in any of the methods described herein. In some embodiments, cells expressing a chimeric receptor are “pooled,” i.e., two or more groups of cells express two or more different CARs. Two or more cells expressing different CARs can be administered or sequentially. In some embodiments, epitopes of CD38 are targeted by cytotoxic agents. In some embodiments, the CARs targeting CD38 are expressed in the same immune cell (i.e., a bispecific immune cell). Such cells can be used in any of the methods described herein. In some embodiments, cells expressing chimeric receptors targeting CD38 “pooled,” i.e., two or more groups of cells express two or more different CARs. Two or more groups of cells expressing CARs targeting CD38 can be administered concurrently or sequentially.

In addition to an epitope-binding fragment described herein, a CAR may further include one or more of the following: a hinge domain (e.g., CD28 hinge, IgG4 hinge, or CD8alpha hinge), a transmembrane domain (e.g., CD28 TM, CD8alpha TM, 4-1BB TM), a co-stimulatory domain (e.g., CD28z, 4-1BB, ICOS, OX40), a cytoplasmic signaling domain (e.g., CD3z), and combinations thereof.

In some embodiments, the hinge domain may be located between the epitope binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the epitope binding fragment relative to another domain of the chimeric receptor can be used. The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain, or at least a portion thereof, is a hinge domain of a naturally occurring protein. In some embodiments, the hinge domain is of CD8alpha or CD28. In some embodiments, the hinge domain is a portion of the hinge domain of CD8alpha, e.g., a fragment containing at least 15 (e.g., 20, 25, 30, 35, or 40) consecutive amino acids of the hinge domain of CD8alpha or CD28.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

In some embodiments, the CARs described herein may include one or more transmembrane domain(s), which can be in any form known in the art. As used herein, a “transmembrane domain” refers to any protein structure that is thermodynamically stable in a cell membrane, preferably a eukaryotic cell membrane. Transmembrane domains compatible for use in the CARs used herein may be obtained from a naturally occurring protein. Alternatively, the transmembrane domain may be a synthetic, non-naturally occurring protein segment, e.g., a hydrophobic protein segment that is thermodynamically stable in a cell membrane.

Transmembrane domains are classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). In some embodiments, the transmembrane domain is a single-pass transmembrane domain. In some embodiments, the transmembrane domain is a single-pass transmembrane domain that orients the N terminus of the chimeric receptor to the extracellular side of the cell and the C terminus of the chimeric receptor to the intracellular side of the cell. In some embodiments, the transmembrane domain is obtained from a single pass transmembrane protein. In some embodiments, the transmembrane domain is of CD28 or 4-1BB or CD8alpha.

In some embodiments, the CARs described herein include one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

In some embodiments, the CARs described herein include more than one (at least 2, at least 3, at least 4, or even more) co-stimulatory signaling domains. In some embodiments, the chimeric receptor comprises more than one co-stimulatory signaling domains obtained from different costimulatory proteins. In some embodiments, the chimeric receptor does not comprise a co-stimulatory signaling domain.

In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the CARs described herein. The type(s) of co-stimulatory signaling domain is selected based on factors such as the type of the immune cells in which the CARs would be expressed (e.g., primary T cells, T cell lines, NK cell lines) and the desired immune effector function (e.g., cytotoxicity). Examples of co-stimulatory signaling domains for use in the CARs can be the cytoplasmic signaling domain of co-stimulatory proteins, including, without limitation, CD27, CD28((CD28z), 4-1BB, OX40, CD30, ICOS, CD2, CD7, LIGHT, NKG2C, B7-H3.

In some embodiments, the chimeric receptors described herein comprise one or more cytoplasmic signaling domain(s). Any cytoplasmic signaling domain can be used in the chimeric receptors described herein. In general, a cytoplasmic signaling domain relays a signal, such as interaction of an extracellular ligand-binding domain with its ligand, to stimulate a cellular response, such as inducing an effector function of the cell (e.g., cytotoxicity). In some embodiments, the cytoplasmic signaling domain is from CD3zeta (CD3z).

In some embodiments, provided herein are CAR constructs targeting CD38 or CD38 plus other gene(s). The construct can further include at least a hinge domain (e.g., from CD28, CD8alpha, or an antibody), a transmembrane domain (e.g., from CD28), one or more co-stimulatory domains (from one or more of CD28z) and a cytoplasmic signaling domain (e.g., from CD3z), or a combination thereof. In some examples, the methods described herein involve administering to a subject a population of genetically engineered cells (e.g., HSPCs or T cells) (engineered to have a mutant CD38 or CD38 plus other gene(s) such as those disclosed in WO 2023/159136) and/or an immune cell expressing a CAR that targets CD38 or CD38 plus other gene(s), respectively, which may further comprise at least a hinge domain (e.g., from CD28, CD8alpha, or an antibody), a transmembrane domain (e.g., from CD28), one or more co-stimulatory domains (from one or more of CD28z) and a cytoplasmic signaling domain (e.g., from CD3z), or combination thereof. In some embodiments, the administered immunotherapeutic product is a combination of immune cells expressing individual chimeric receptor that targets CD38.

Any of the CARs described herein can be prepared by routine methods, such as recombinant technology. Methods for preparing the chimeric receptors herein involve generation of a nucleic acid that encodes a polypeptide comprising each of the domains of the chimeric receptors, including the epitope binding fragment and optionally, the hinge domain, the transmembrane domain, at least one co-stimulatory signaling domain, and the cytoplasmic signaling domain. In some embodiments, nucleic acids encoding the components of a chimeric receptor are joined together using recombinant technology.

Additionally, any of the CARs can be expressed in immune cells and administered to a human subject by routine methods. For example, T cells can be either derived from T cells in a subject's own blood (autologous) or derived from the T cells of another healthy donor (allogeneic). Once isolated from a subject, these T cells are genetically engineered to express a specific CAR, which programs them to target an antigen that is present on the surface of tumors. The CAR-T cells are then infused, by customary practice, into the subject.

In some embodiments, a CAR is at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs: 64 or 66, wherein the CAR retains its ability to bind to CD38.

In some embodiments, a CAR includes one or more epitope binding fragments comprises the one or more epitope binding fragments from SEQ ID NO: 64 or 66.

In some embodiments, the cell-surface lineage-specific protein is CD38 and the epitope binding fragment comprises the following CDR sequences: VH CDR1: SFAMS (SEQ ID NO: 57), VH CDR2: AISGSGGGTYYADSVK (SEQ ID NO: 58); VH CDR3: DKILWFGEPVFDY (SEQ ID NO: 59); VL CDR1: RASQSVSSYLAW (SEQ ID NO: 60); VL CDR2: DASNRAT (SEQ ID NO: 61); VL CDR3: QQRSNWPPTF (SEQ ID NO: 62).

Exemplary CAR sequences are provided below:

SEQ ID NO: 63 anti-CD38 CAR-1 (CD8a hinge, CD28 TM, CD28z, CD3z)
nucleotide sequence:
ATGCTCTTGTTGGTGACGAGTCTCCTGCTGTGTGAACTGCCGCACCCAGCATTTCTTT
TGATTCCGGAGGTGCAGCTGTTGGAGTCTGGGGGAGGCTTGGTACAGCCTGGGGGG
TCCCTGAGACTCTCATGTGCAGTCTCTGGATTCACCTTTAACAGCTTTGCCATGAGCT
GGGTCCGCCAGGCTCCAGGGAAGGGGCTGGAGTGGGTCTCAGCTATTAGTGGTAGT
GGTGGTGGCACATACTACGCAGACTCCGTGAAGGGCCGGTTCACCATCTCCAGAGA
CAATTCCAAGAACACGCTGTATCTGCAAATGAACAGCCTGAGAGCCGAGGACACGG
CCGTATATTTCTGTGCGAAAGATAAGATTCTCTGGTTCGGGGAGCCCGTCTTTGACT
ACTGGGGCCAGGGAACCCTGGTCACCGTCTCCTCAGCCTCCGGAGGCGGAGGCTCC
GGTGGGGGAGGATCTGGGGGAGGCGGAAGCGAAATTGTGTTGACACAGTCTCCAGC
CACCCTGTCTTTGTCTCCAGGGGAAAGAGCCACCCTCTCCTGCAGGGCCAGTCAGAG
TGTTAGCAGCTACTTAGCCTGGTACCAACAGAAACCTGGCCAGGCTCCCAGGCTCCT
CATCTATGATGCATCCAACAGGGCCACTGGCATCCCAGCCAGGTTCAGTGGCAGTG
GGTCTGGGACAGACTTCACTCTCACCATCAGCAGCCTAGAGCCTGAAGATTTTGCAG
TTTATTACTGTCAGCAGCGTAGCAACTGGCCTCCGACGTTCGGCCAAGGGACCAAGG
TGGAAATCAAAACCACAACGCCCGCTCCTCGGCCACCGACGCCAGCGCCAACTATT
GCGAGTCAGCCTCTCAGTCTGCGACCTGAGGCTTGTCGACCAGCAGCCGGAGGCGC
AGTGCACACGAGGGGGCTGGACTTCGCCTGTGATGCTAGCATGTTCTGGGTGCTGGT
GGTGGTCGGAGGCGTGCTGGCCTGCTACAGCCTGCTGGTCACCGTGGCCTTCATCAT
CTTTTGGGTCCGCAGCAAGCGGAGCAGAGGCGGCCACAGCGACTACATGAACATGA
CCCCTAGACGGCCTGGCCCCACCAGAAAGCACTACCAGCCCTACGCCCCTCCCCGG
GACTTTGCCGCCTACAGAAGCCGGGTGAAGTTCAGCAGAAGCGCCGACGCCCCTGC
CTACCAGCAGGGCCAGAATCAGCTGTACAACGAGCTGAACCTGGGCAGAAGGGAA
GAGTACGACGTCCTGGATAAGCGGAGAGGCCGGGACCCTGAGATGGGCGGCAAGC
CTCGGCGGAAGAACCCCCAGGAAGGCCTGTATAACGAACTGCAGAAAGACAAGAT
GGCCGAGGCCTACAGCGAGATCGGCATGAAGGGCGAACGGAGGCGGGGCAAGGGC
CACGACGGCCTGTATCAGGGCCTGTCCACCGCCACCAAGGATACCTACGACGCCCT
GCACATGCAGGCCCTGCCCCCAAGGTGA
SEQ ID NO: 64 anti-CD38 CAR-1 (CD8a hinge, CD28 TM, CD28z, CD3z) amino
acid sequence (CDRs are bold):
MLLLVTSLLLCELPHPAFLLIPEVQLLESGGGLVQPGGSLRLSCAVSGFTFNSFAMSWVR
QAPGKGLEWVSAISGSGGGTYYADSVKGRFTISRDNSKNTLYLQMNSLRAEDTAVYFC
AKDKILWFGEPVFDYWGQGTLVTVSSASGGGGSGGGGSGGGGSEIVLTQSPATLSLSP
GERATLSCRASQSVSSYLAWYQQKPGQAPRLLIYDASNRATGIPARFSGSGSGTDFTLTI
SSLEPEDFAVYYCQQRSNWPPTFGQGTKVEIKTTTPAPRPPTPAPTIASQPLSLRPEACRP
AAGGAVHTRGLDFACDASMFWVLVVVGGVLACYSLLVTVAFIIFWVRSKRSRGGHSD
YMNMTPRRPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMKGERRRGK
GHDGLYQGLSTATKDTYDALHMQALPPR
SEQ ID NO: 65 anti-CD38 CAR-2 (IgG4 hinge, CD28 TM, CD28z, CD3z)
nucleotide sequence:
ATGCTCTTGTTGGTGACGAGTCTCCTGCTGTGTGAACTGCCGCACCCAGCATTTCTTT
TGATTCCGGAGGTGCAGCTGTTGGAGTCTGGGGGAGGCTTGGTACAGCCTGGGGGG
TCCCTGAGACTCTCATGTGCAGTCTCTGGATTCACCTTTAACAGCTTTGCCATGAGCT
GGGTCCGCCAGGCTCCAGGGAAGGGGCTGGAGTGGGTCTCAGCTATTAGTGGTAGT
GGTGGTGGCACATACTACGCAGACTCCGTGAAGGGCCGGTTCACCATCTCCAGAGA
CAATTCCAAGAACACGCTGTATCTGCAAATGAACAGCCTGAGAGCCGAGGACACGG
CCGTATATTTCTGTGCGAAAGATAAGATTCTCTGGTTCGGGGAGCCCGTCTTTGACT
ACTGGGGCCAGGGAACCCTGGTCACCGTCTCCTCAGCCTCCGGAGGCGGAGGCTCC
GGTGGGGGAGGATCTGGGGGAGGCGGAAGCGAAATTGTGTTGACACAGTCTCCAGC
CACCCTGTCTTTGTCTCCAGGGGAAAGAGCCACCCTCTCCTGCAGGGCCAGTCAGAG
TGTTAGCAGCTACTTAGCCTGGTACCAACAGAAACCTGGCCAGGCTCCCAGGCTCCT
CATCTATGATGCATCCAACAGGGCCACTGGCATCCCAGCCAGGTTCAGTGGCAGTG
GGTCTGGGACAGACTTCACTCTCACCATCAGCAGCCTAGAGCCTGAAGATTTTGCAG
TTTATTACTGTCAGCAGCGTAGCAACTGGCCTCCGACGTTCGGCCAAGGGACCAAGG
TGGAAATCAAAGAGTCTAAGTACGGACCGCCCTGCCCCCCTTGCCCTGCTAGCATGT
TCTGGGTGCTGGTGGTGGTCGGAGGCGTGCTGGCCTGCTACAGCCTGCTGGTCACCG
TGGCCTTCATCATCTTTTGGGTCCGCAGCAAGCGGAGCAGAGGCGGCCACAGCGAC
TACATGAACATGACCCCTAGACGGCCTGGCCCCACCAGAAAGCACTACCAGCCCTA
CGCCCCTCCCCGGGACTTTGCCGCCTACAGAAGCCGGGTGAAGTTCAGCAGAAGCG
CCGACGCCCCTGCCTACCAGCAGGGCCAGAATCAGCTGTACAACGAGCTGAACCTG
GGCAGAAGGGAAGAGTACGACGTCCTGGATAAGCGGAGAGGCCGGGACCCTGAGA
TGGGCGGCAAGCCTCGGCGGAAGAACCCCCAGGAAGGCCTGTATAACGAACTGCAG
AAAGACAAGATGGCCGAGGCCTACAGCGAGATCGGCATGAAGGGCGAACGGAGGC
GGGGCAAGGGCCACGACGGCCTGTATCAGGGCCTGTCCACCGCCACCAAGGATACC
TACGACGCCCTGCACATGCAGGCCCTGCCCCCAAGGTGA
SEQ ID NO: 66 anti-CD38 CAR-2 (IgG4 hinge, CD28 TM, CD28z, CD3z) amino
acid sequence (CDR's are bold):
MLLLVTSLLLCELPHPAFLLIPEVQLLESGGGLVQPGGSLRLSCAVSGFTFNSFAMSWVRQAP
GKGLEWVSAISGSGGGTYYADSVKGRFTISRDNSKNTLYLQMNSLRAEDTAVYFCAKDKILW
FGEPVFDYWGQGTLVTVSSASGGGGSGGGGSGGGGSEIVLTQSPATLSLSPGERATLSCRAS
QSVSSYLAWYQQKPGQAPRLLIYDASNRATGIPARFSGSGSGTDFTLTISSLEPEDFAVYYCQ
QRSNWPPTFGQGTKVEIKESKYGPPCPPCPASMFWVLVVVGGVLACYSLLVTVAFIIFW
VRSKRSRGGHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDKMAEAYSE
IGMKGERRRGKGHDGLYQGLSTATKDTYDALHMQALPPR
III. Methods of Treating a Subject

The genetically engineered cells (e.g., HSPCs or T cells) can be administered to a human subject in need of the treatment, either taken alone or in combination of one or more cytotoxic agents that target one or more cell-surface antigens as described herein. Since the cells are genetically edited in the genes of the one or more cell-surface antigens, the cells and/or descendant cells thereof would express the one or more cell-surface antigens in mutated form (e.g., but functional) such that they can escape being targeted by the cytotoxic agents, for example, CAR-T cells.

Thus, the present disclosure provides methods for treating a condition that typically affects the wild-type form of the engineered cells, the method including administering to a human subject in need thereof (i) a population of the genetically engineered cells (e.g., HSPCs or T cells) described herein, and optionally (ii) a cytotoxic agent (e.g., CAR-T cells) that target a cell-surface antigen, the gene of which is genetically edited in the cells such that the cytotoxic agent does not target the wild-type form of the engineered cells or descendant cells thereof In embodiments where both (i) and (ii) are administered, the administration of (i) and (ii) can be concurrently or in any order. In some embodiments, the cytotoxic agents and/or the cells can be mixed with a pharmaceutically acceptable carrier to form a pharmaceutical composition, which is also within the scope of the present disclosure.

To perform the methods described herein, an effective amount of the genetically engineered cells (e.g., HSPCs or T cells) can be administered to a human subject in need of the treatment. Optionally, the genetically engineered cells can be co-used with a cytotoxic agent as described herein. In some embodiments, the subject is a human patient having a hematopoietic malignancy.

As used herein the term “effective amount” can be used interchangeably with the term “therapeutically effective amount.” Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner.

As described herein, the genetically engineered cells expressing chimeric receptors can be autologous to the subject, i.e., the cells are obtained from the subject in need of the treatment, manipulated such that the cells do not bind the cytotoxic agents, and then administered to the same subject. Administration of autologous cells to a subject can result in reduced rejection of the host cells as compared to administration of non-autologous cells. For example, HSPCs or T cells are obtained from a biological sample from a subject, the HSPCs or T cells are genetically engineered, and the genetically engineered HSPCs or T cells are administered to the same subject. In some instances, the HSPCs or T cells are obtained from a biological sample, wherein the biological sample is bone marrow cells, blood, cord blood cells, or mobilized peripheral blood-derived CD34+ hematopoietic stem and progenitor cells.

Alternatively, the host cells are allogeneic cells (i.e., the cells are obtained from a first subject), genetically engineered, and then administered to a second subject that is different from the first subject but of the same species. For example, allogeneic immune cells can be derived from a human donor and administered to a human recipient who is different from the donor. In some embodiments, the genetically engineered cells have been further genetically engineered to reduce host-versus-graft effects. For example, in some embodiments, immune cells and/or genetically engineered cells can be subjected to gene editing or silencing methods to reduce or eliminate expression of one or more proteins involved in inducing host immune responses.

A typical amount of cells(i.e., immune cells or genetically engineered cells of the present disclosure) administered to a subject can be, for example, in a range of 10⁶ to 10¹¹ cells. In some embodiments, it can be desirable to administer fewer than 10⁶ cells to the subject. In some embodiments, it can be desirable to administer more than 10¹¹ cells to the subject. In some embodiments, one or more doses of cells includes 10⁶ cells to 10¹¹ cells, 10⁷ cells to 10¹⁰ cells, 10⁸ cells to 10⁹ cells, 10⁶ cells to 10⁸ cells, 10⁷ cells to 10⁹ cells, 10⁷ cells to 10¹⁰ cells, 10⁷ cells to 10¹¹ cells, 10⁸ cells to 10¹⁰ cells, 10⁸ cells to 10¹¹ cells, 10⁹ cells to 10¹⁰ cells, 10⁹ cells to 10¹¹ cells, or 10¹⁰ cells to 10¹¹ cells.

In some embodiments, the methods described herein involve administering a population of genetically engineered cells (e.g., HSPCs or T cells) to a subject and administering one or more immunotherapeutic agents (e.g., cytotoxic agents). As will be appreciated by one of ordinary skill in the art, the immunotherapeutic agents can be of the same or different type (e.g., therapeutic antibodies, populations of immune cells expressing chimeric antigen receptor(s), and/or antibody-drug conjugates).

In some embodiments, the cytotoxic agent including an epitope binding fragment that binds an epitope of a cell-surface protein (e.g., immune cells expressing a CAR as described herein) is administered prior to administration of the genetically engineered cells. This can be at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months or more prior to administration of the genetically engineered cells.

Alternatively, in some embodiments, the genetically engineered cells are administered prior to the cytotoxic agent including an epitope binding fragment that binds an epitope of the cell-surface protein (e.g., immune cells expressing a CAR as described herein). This can be at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 3 months, at least 4 months, at least 5 months, at least 6 months, or more prior to administration of the cytotoxic agent including an epitope binding fragment that binds to an epitope of the cell-surface protein.

In some embodiments, the cytotoxic agent targeting the cell-surface protein and the population of genetically engineered cells (HSPCs or T cells) are administered at substantially the same time. In some embodiments, the cytotoxic agent targeting the cell-surface protein is administered and the patient is assessed for a period of time, after which the population of genetically engineered cells is administered. In some embodiments, the population of genetically engineered cells is administered and the patient is assessed for a period of time, after which the cytotoxic agent targeting the cell-surface protein is administered.

Also within the scope of the present disclosure are multiple administrations (e.g., doses) of the cytotoxic agents and/or populations of genetically engineered cells. In some embodiments, the cytotoxic agents and/or populations of genetically engineered cells are administered to the subject once. In some embodiments, cytotoxic agents and/or populations of genetically engineered cells are administered to the subject more than once (e.g., at least 2, at least 3, at least 4, at least 5, or more times). In some embodiments, the cytotoxic agents and/or populations of genetically engineered cells are administered to the subject at a regular interval, e.g., every six months.

Examples of routes of administration include intravenous, infusion, intradermal, subcutaneous, oral (e.g., inhalation), transdermal (topical), transmucosal, and rectal administration.

Any of the methods described herein can be for the treatment of a hematological malignancy in a subject. The term “treat” or “treatment” or “treating” or “to treat” as used herein refers to therapeutic measures that aim to relieve, slow down progression of, lessen symptoms of, and/or halt progression of a pathologic condition or disorder. Thus, those in need of treatment include those already with the disorder. Herein, treating a cancer includes stabilizing progression of the cancer, slowing down progression of the cancer, halting progression of the cancer, shrinking the cancer size, or increasing the overall survival of the subject diagnosed with the cancer. Methods of assessing the progression of a cancer are known in the art and include, for example, evaluation of target lesions using imaging (e.g., X-ray, computerized tomography scan, magnetic resonance imaging, caliper measurement, or positron emission tomography scan), cytology or histology, or expression of tumor marker(s).

In some embodiments, the human subject has a hematological condition, such as a hematopoietic malignancy. As used herein, a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia. Examples of hematological conditions other than hematopoietic malignancies include, without limitation, hemoglobinopathies such as: sickle cell disease, thalassemias or primary immunodeficiencies such as SCID.

In some embodiments, cells involved in the hematopoietic malignancy are resistant to conventional or standard therapeutics used to treat the malignancy. For example, the cells (e.g., cancer cells) can be resistant to a chemotherapeutic agent and/or CAR-T cells used to treat the malignancy.

In some instances, the hematopoietic malignancies include high-risk acute myeloid leukemia (AML) or multiple myeloma.

### IV. Compositions and Kits

Any of the immune cells expressing chimeric receptors and/or genetically engineered cells (e.g., HSPCs or T cells) described herein can be administered in a pharmaceutically acceptable carrier as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and can comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

Also within the scope of the present disclosure are kits for use in treating a hematological condition (e.g., a hematopoietic malignancy). Such a kit can comprise the genetically engineered cells (e.g., HSPCs or T cells), and optionally one or more cytotoxic agents targeting cell-surface antigens, the genes of which are edited in the hematopoietic cells. Such kits can include a container that contains a first pharmaceutical composition that includes any of the genetically engineered cells (e.g., HSPCs or T cells) as described herein, and optionally one or more additional containers that contain one or more cytotoxic agents (e.g., immune cells expressing chimeric receptors described herein) targeting the cell-surface antigens as also described herein.

In some embodiments, the kit can include instructions for use in any of the methods described herein. The included instructions can comprise a description of administration of the genetically engineered cells (e.g., HSPCs or T cells) and optionally descriptions of administration of the one or more cytotoxic agents to a subject to achieve the intended activity in a subject. The kit can further comprise a description of selecting a subject suitable for treatment based on identifying whether the subject is in need of the treatment. In some embodiments, the instructions comprise a description of administering the genetically engineered cells (e.g., HSPCs or T cells) and optionally the one or more cytotoxic agents to a subject who is in need of the treatment.

The instructions relating to the use of the genetically engineered cells (e.g., HSPCs or T cells) and optionally the cytotoxic agents described herein generally include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit can have a sterile access port (for example, the container can be an intravenous solution bag or a vial having a stopper pierceable by a hypodermic injection needle). The container can also have a sterile access port. At least one active agent in the pharmaceutical composition is a chimeric receptor variants as described herein.

Kits optionally can provide additional components such as buffers and interpretive information. Normally, the kit comprises a container and a label or package insert(s) on or associated with the container. In some embodiment, the disclosure provides articles of manufacture comprising contents of the kits described above.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

hCD38 Base Editing and Anti-CD38 CAR-T Production.

### Example 1: Epitope Editing of hCD38

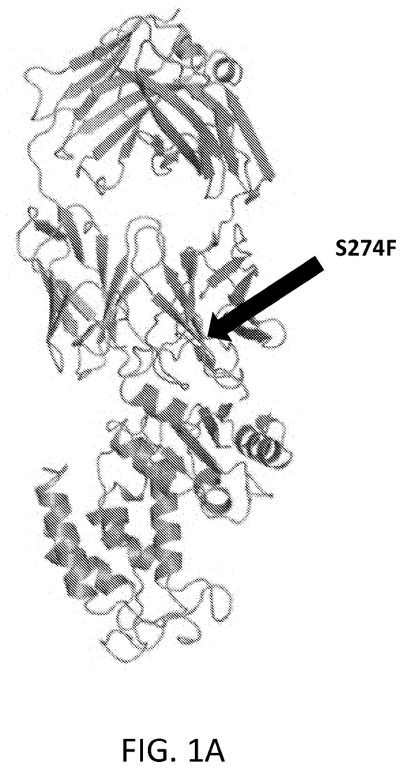

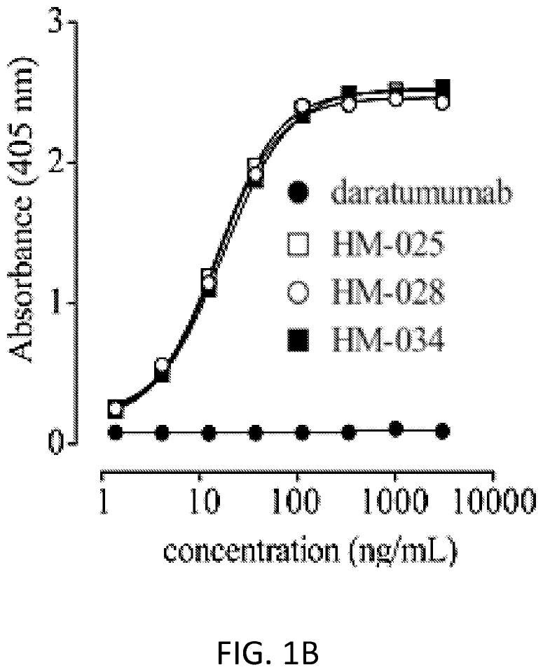

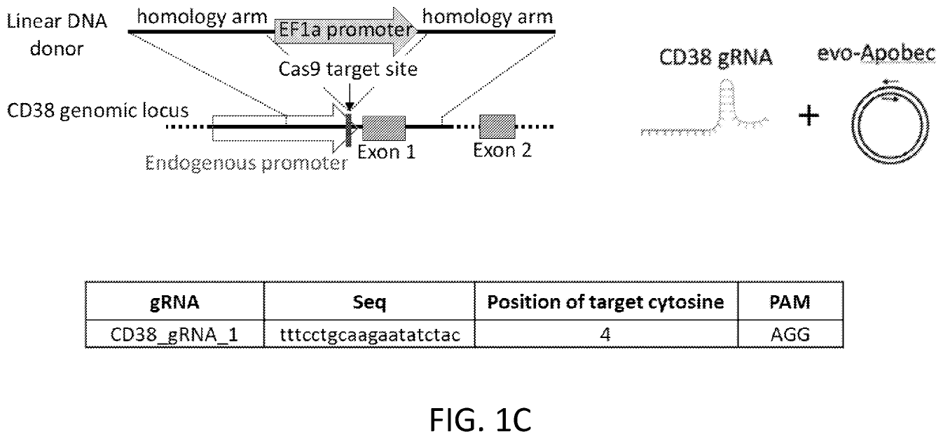

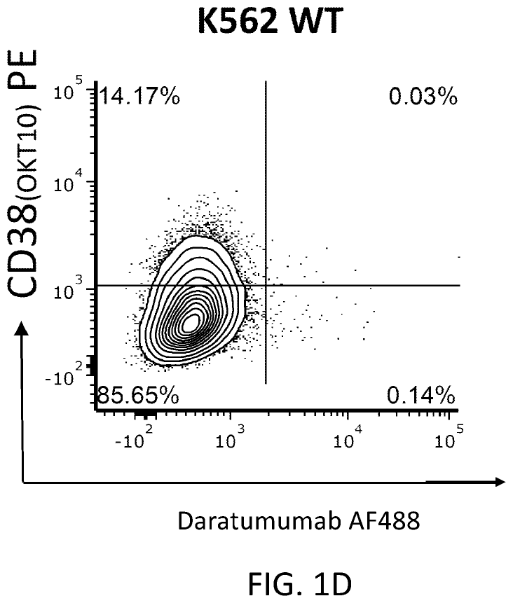

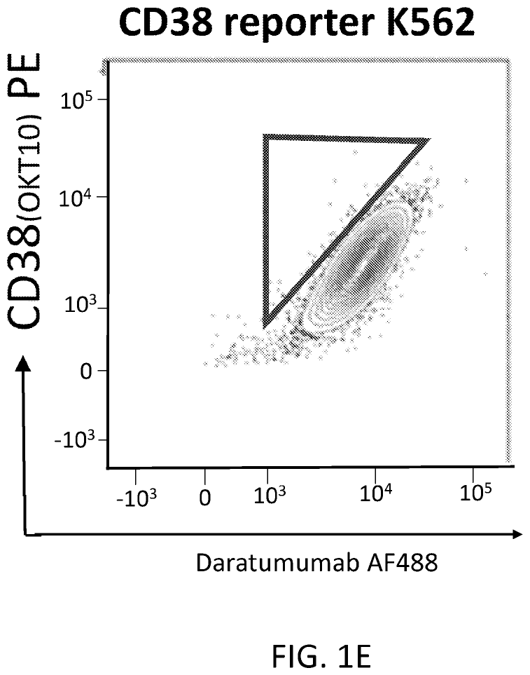

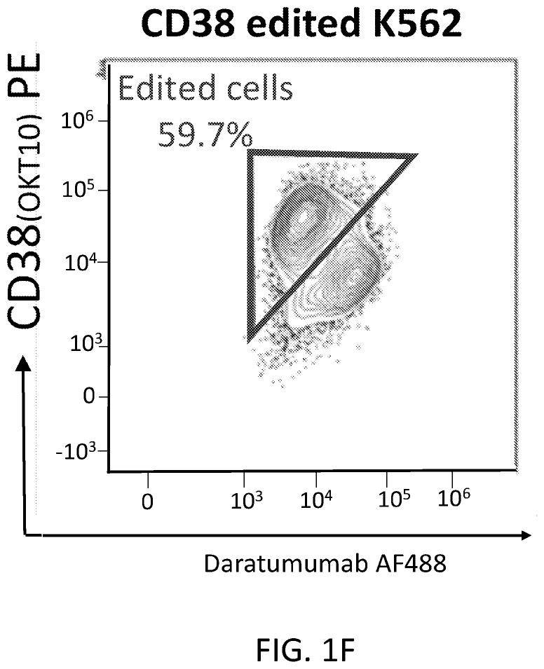

Human CD38 is a type II transmembrane glycoprotein expressed by premature hematopoietic cells, lost by mature cells, and re-expressed on activated lymphocytes, such as T cells, B cells, dendritic cells, and natural killer cells. Based on data available disclosed by Michel de Weers et al. in J. Immunol., 186 (3): 1840-1848 (2011) (doi.org/10.4049/jimmunol.1003032), one orthologous mutation from cynomolgus monkey (MacacaFascicularis) on CD38 protein (replacing serine at position 274 for phenylalanine (S274F)) that can efficiently impair the binding of the antibody daratumumab on CD38 glycoprotein was identified (see FIG. 1A and FIG. 1B).

A genetic engineering strategy was designed for inducing the S274F mutation on human cells through a base editing approach. For testing this hypothesis, a reporter cell line overexpressing the CD38 from its endogenous locus was generated by integrating with a homologous mediated recombination approach an artificial promoter upstream to the translation starting site of the gene (FIG. 1C). Subsequently, the editing strategy was tested by delivering to the reporter cell line (K562 cells) both the CD38_gRNA_1 (SEQ ID NO:11) and a plasmid expressing the Cytidine Base Editor 4 (available from Addgene, Cambridge, MA) through electroporation using Lonza 4D-NUCLEOFECTORr System in SF solution according to the manufacturer instructions (Lonza, Basel, Switzerland). 500 ng of base editor expression plasmid and 300-360 pmol of sgRNA (available from Integrated DNA Technologies, Coralville, IA) were included in the electroporation reaction. The editing strategy generated a CD38 variant which lacked recognition by an ALEXA FLUOR 488 conjugated daratumumab antibody (available from Selleckchem, Houston, TX) but retained surface expression by staining with a Phycoerythrin (PE)-conjugated control antibody OKT10 (available from Leinco Technologies, Fenton, MO) in a Fluorescence Activated Cell Sorting (FACS) assay (FIGS. 1D, 1E, and 1F).

### Example 2: Design and Production of Daratumumab-Based Anti-CD38 CAR-T

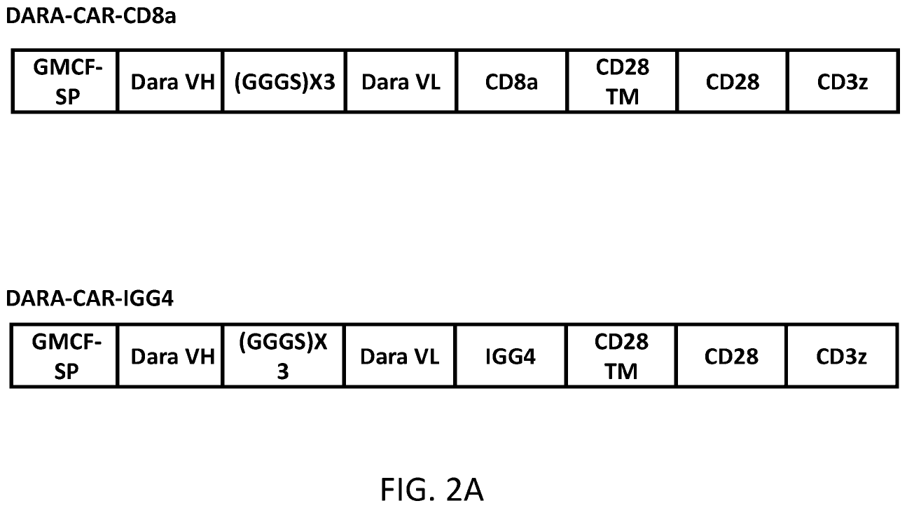

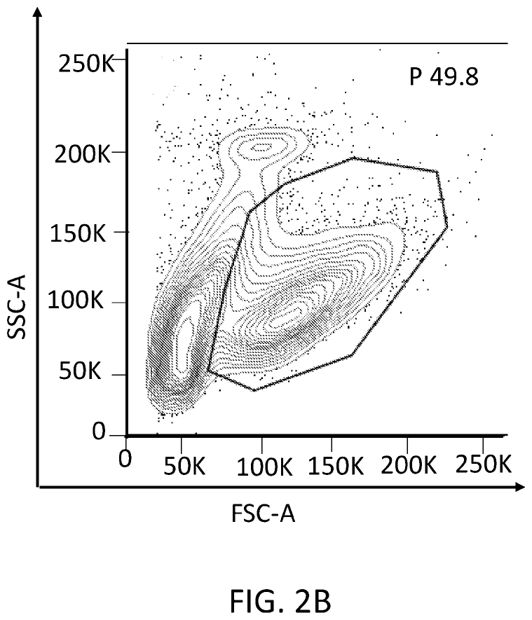

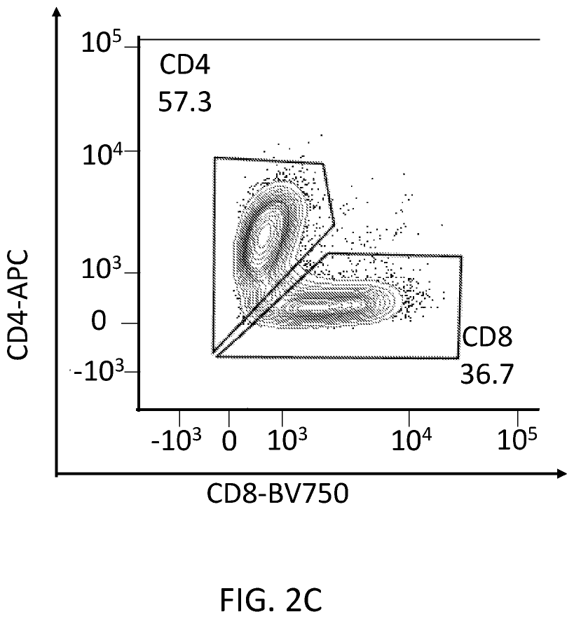

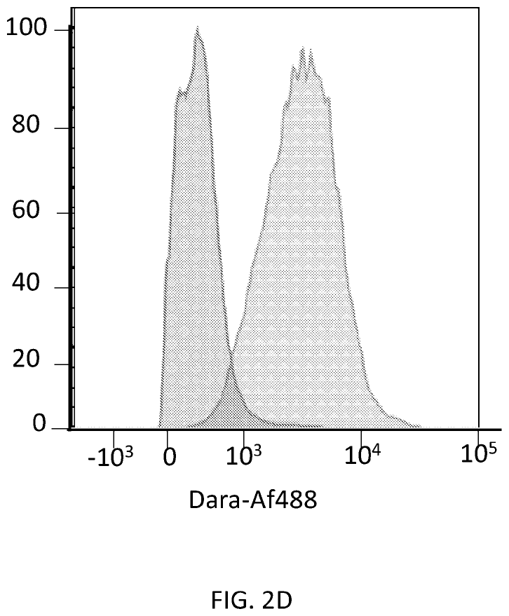

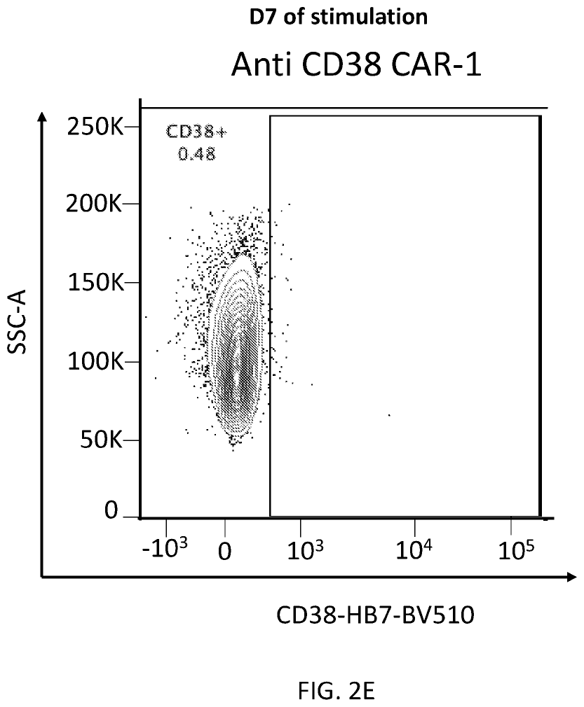

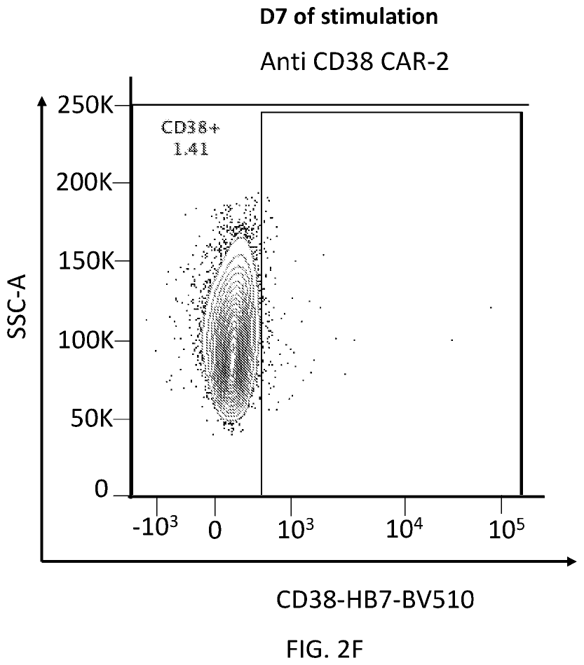

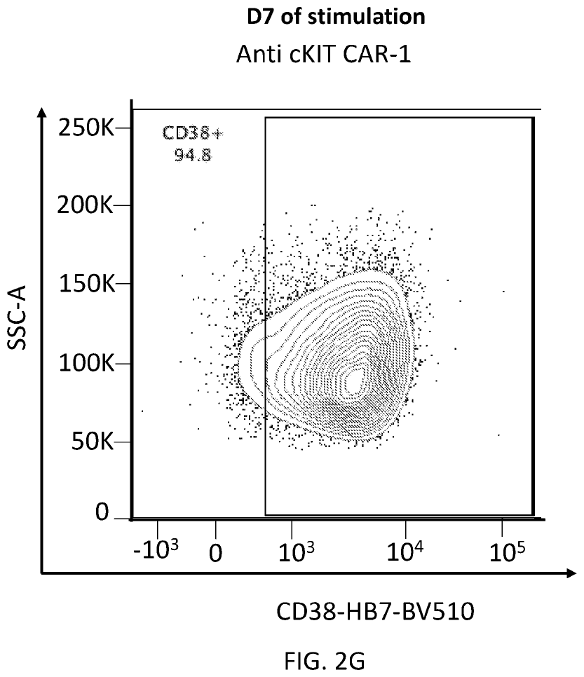

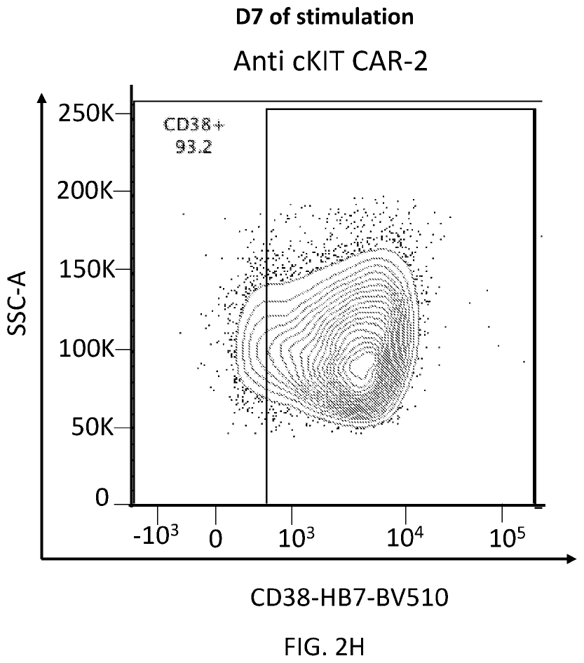

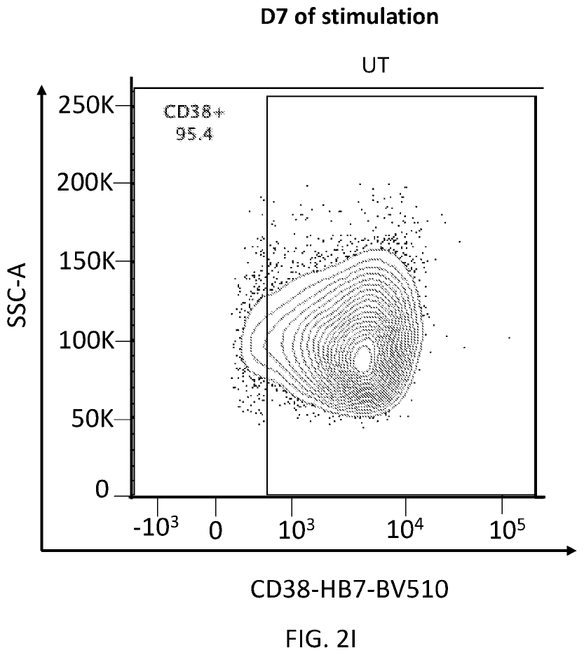

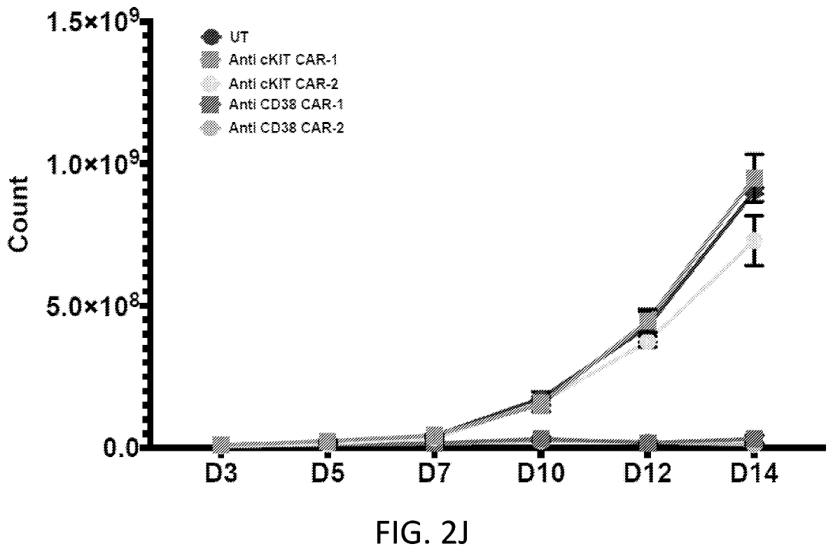

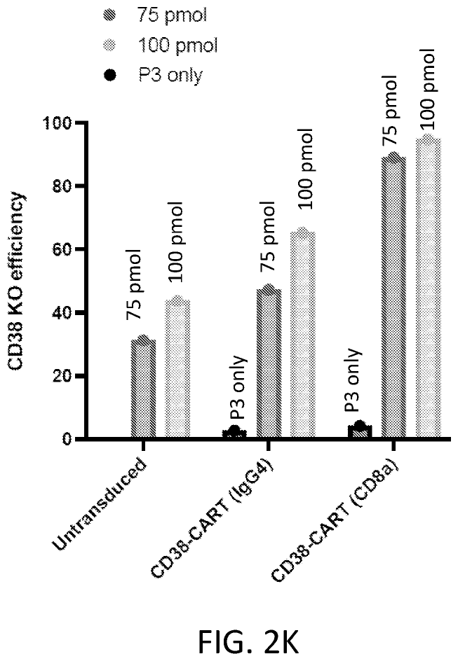

Rationally designed anti-CD38 chimeric antigen receptors may result in potent antitumor efficacy. FIG. 2A is a schematic of two second generation chimeric antigen receptors targeting human CD38 (SEQ ID NOs: 63-66). Both the constructs encode the variable heavy chain and the variable light chain of daratumumab assembled through a GGGSx3 linker (GGGSGGGSGGGS); the only difference between the two constructs is the hinge domain (CD8a and IgG4 respectively for CAR1 and CAR2). Two III-generation lentiviral constructs expressing the second generation, CD38-specific chimeric antigen receptors under a constitutive hPGK promoter were cloned using synthesized dsDNA fragments (IDT gBlocks). An antisense cassette expressing a truncated variant of the human EGFR cDNA under a minimal-CMV promoter was included to serve as marker of transduction and safety switch for in vivo depletion by using anti-EGFR antibody Cetuximab. VSV-G pseudotyped self-inactivating lentiviral particles (LVs) were prepared according to published methods (epfl.ch/labs/tronolab/wp-content/uploads/2019/06/LV_production.pdf) by calcium-phosphate transient co-transfection of 5 plasmids in HEK-293T (human embryonic kidney) cells (transfer vector, pMD2, pMDL-RRE, pREV and pAdvantage plasmids). Viral particles-containing supernatants were concentrated 500-fold by ultracentrifugation (20000 rpm at 20° C. for 2 hours) and resuspended in phosphate buffered saline (PBS). Concentrated LVs were titrated by transducing HEK-293T cells at different concentrations and calculating the transduction efficiency by flow cytometry or droplet digital PCR (ddPCR).

Peripheral blood mononuclear cells (PBMC) were isolated by ficoll gradient separation from whole blood. T-cells were magnetically sorted using the human Pan T Cell Isolation Kit (available from Miltenyi Biotec,Cologne, Germany). Either freshly isolated or thawed T cells were incubated with CD3-CD28 DYNABEADS (Gibco 11131D, available from Thermofisher Scientific, Waltham, MA) at 3:1 bead:T-cell ratio, and cultured at 1 M/mL in IMDM (Iscove's Modified Dulbecco's Medium, available from Thermofisher Scientific, product number 12440053) supplemented with 10% fetal bovine serum (FBS), 1%

Penicillin/Streptomycin (P/S), human IL-7 (5 ng/mL, available from PeproTech, Cedarbrook, NJand human IL-15 (5 ng/mL available from PeproTech). Forty-eight hours (h) after the start of the stimulation with the DYNABEADS, T cells were transduced at a multiplicity of infection (MOI) 5 to MOI 10, depending on the experiment with lentiviral particles encoding for the CAR of choice. DYNABEADS were removed from the culture by magnetic separation at day 7 since the start of the stimulation, and T cells were expanded for an additional 5-7 days in IMDM supplemented with 10% FBS, 1% P/S, human IL-7 (5 ng/mL, Peprotech) and human IL-15 (5 ng/mL, Peprotech). T cell phenotype and transduction efficiency (by EGFR surface staining) was evaluated periodically by flow cytometry. Expanded CAR-T cells or untransduced T cells were either used for killing assays, in vivo administration, or vitally frozen after 12-14 days since the start of the stimulation.

FIGS. 2B, 2C, and 2D show the FACS plot of the CD38 expression on activated T cells at day 3 of stimulation and before CAR transduction. All the T cells uniformly expressed the CD38 glycoprotein. The same evaluation was performed four days later (D7 after CAR transduction; see FIGS. 2E, 2F, 2G, 2H, and 2I). While anti-cKIT CAR-T cells (produced in parallel as a positive control) and the untransduced cells (UT) retain the CD38 expression (see FIGS. 2G, 2H, and 2I), both anti-CD38 CAR-T cells completely lost the surface marker expression (see FIGS. 2E and 2F. One hypothesis is that the loss of expression of CD38 in the CAR-T cells production protocol is due to a fratricide effect. As shown in FIG. 2J, while anti-cKIT CAR-T cells showed growth curve and fold expansion comparable to the untransduced control (UT), the anti-CD38 CAR-T cells did not expand in culture. To test this hypothesis in another experiment, the CD38_gRNA_1 (SEQ ID NO: 11) was used in combination with a CAS9 protein (available from Syntego Corporation, Redwood City, CA) at two different doses (75-100 μmol) for performing a knock out (KO) experiment. The ribonucleoprotein was delivered to the CAR-T cells through electroporation the day after CAR transduction. After 15 days in culture, the KO efficiency was quantified through a tracking of indels by decomposition (TIDE) analysis on Sanger trace (FIG. 2K) showing a positive selection of the KO cells in the presence of the CAR compared to the untransduced control. Here, the CD38_gRNA_1 was used to perform a KO of CD38. It is thought that the C-T base editing induced with the same gRNA may protect the cells from the fratricide effect, thereby improving the growth and effectiveness of the daratumumab based anti-CD38 CAR-T.

In order to test the efficacy of anti-CD38-CAR-T, K562 cells (either unmodified or overexpressing the CD38 receptor variant after promoter substitution) were plated in a 96-well plate (25000 target cells/well). Anti-CD38 CAR-T or untransduced T cells were then co-plated at different effector:target ratios (E:T ratio), typically 5, 2.5, 1.25, 0.625 in the same wells and incubated at 37° C. with 5% carbon dioxide in a humidified incubator. After 4 hours, the culture volume was harvested for flow cytometry analysis by staining with FcR-blocking reagent (available from Miltenyi Biotech) 2/100 microliter (uL)s, 1/100, CD3 APCh7 (available from BioLegend, San Diego, CA) 2/100, CD38 BV510 (Biolegend,). The staining mix included flow counting beads to normalize cell counts (Biolegend). Cells were than washed and resuspended in AnnexinV binding buffer (Biolegend) supplemented with AnnexinV FITC (Biolegend) 3/100 and 7AAD (available from BD Biosciences,San Jose, CA). Samples were analyzed on a 4- or 5-laser BD Fortessa flow cytometer.

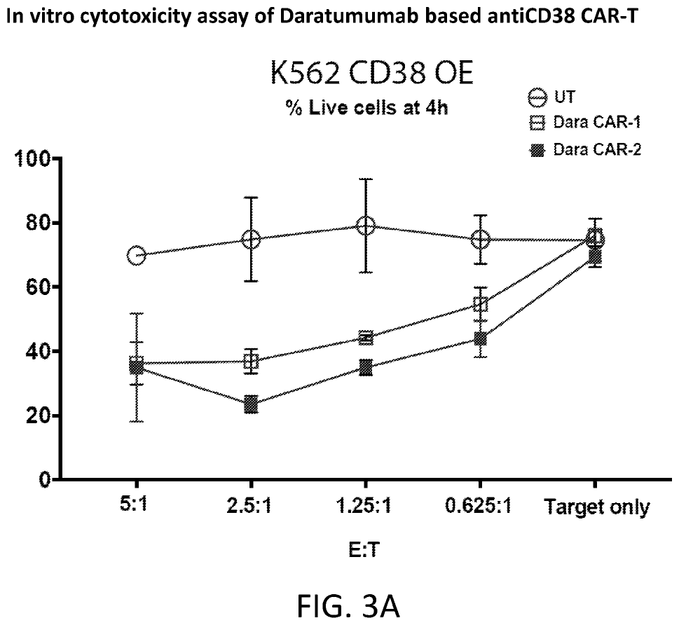

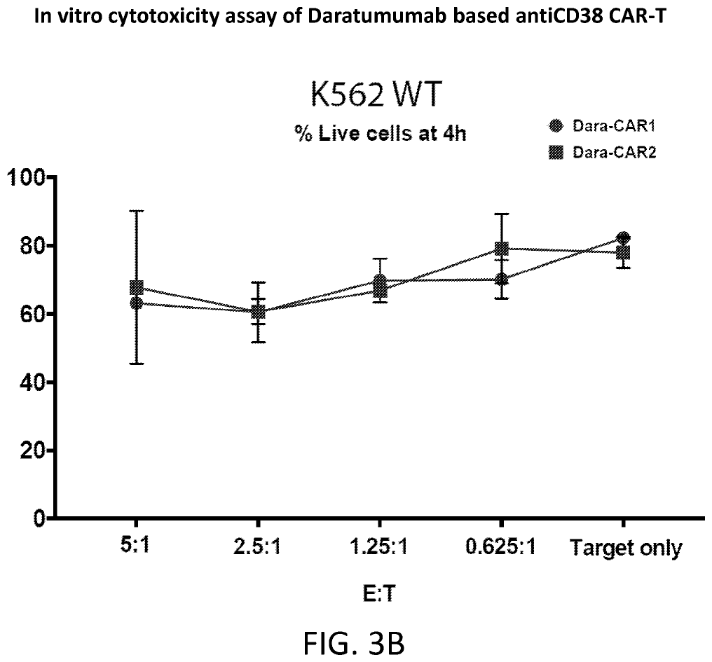

FIG. 3A shows the percentage of CD38 overexpressing live cells (i.e., K562 cells overexpressing CD38) at 4 hours of co-culture with untransduced or anti-CD38-CAR-T cells, estimating the CAR-T cell mediated killing at different effector:target (E:T) ratios. FIG. 3B shows the viability of the control K562 wild-type (WT) cells (not expressing CD38) indicating the specificity of CAR mediated killing.

These examples show that the S274F mutation on human cells can be efficiently introduced through a base editing approach and it can efficiently abrogate the binding of the well-known antibody daratumumab. Two different versions of daratumumab based CAR constructs were designed and produced and their efficacy in an in vitro killing assay assessed. To avoid the fratricide issue, CD38_gRNA_1 sgRNA can be used to either: 1) produce a KO of the CD38 expression (as shown in FIG. 2K); or 2) introduce the S274F on anti-CD38 CAR-T or other CAR-T that could be potentially used in combination.

1. A genetically engineered hematopoietic stem/progenitor cell (HSPC) or T cell, comprising a genetically engineered CD38 gene, wherein the genetically engineered CD38 gene is engineered such that its encoded protein has reduced binding to a therapeutic anti-CD38 antibody.

2. The genetically engineered HSPC or T cell of claim 1, wherein at least one mutation in the genetically engineered CD38 gene results in a polypeptide bearing a mutation at position S274.

3. (canceled)

4. The genetically engineered HSPC or T cell of claim 1, wherein the therapeutic anti-CD38 antibody is daratumumab or an antibody that has the same six complementarity-determining regions (CDRs) as, or is otherwise able to compete for CD38 binding sites with, daratumumab.

5. The genetically engineered HSPC or T cell of claim 1, wherein the genetically engineered CD38 gene encodes a polypeptide comprising the amino acid sequence of SEQ ID NO: 52, or a polypeptide that is at least 85%, at least 90%, at least 95%, at least 96%, at least 97%, at least 98%, or at least 99% identical to the amino acid sequence of SEQ ID NO: 52.

6. (canceled)

7. A population of genetically engineered hematopoietic stem/progenitor cells (HSPCs) or T cells, comprising the genetically engineered HSPC or T cell of claim 1.

8. A pharmaceutical composition comprising the population of genetically engineered hematopoietic stem/progenitor cells or T cells of claim 7 and a pharmaceutically acceptable carrier.

9. A kit comprising the population of genetically engineered hematopoietic stem/progenitor cells or T cells of claim 7, and optionally one or more cytotoxic agents targeting cell-surface antigens, the genes of which are edited in the hematopoietic stem/progenitor cells or T cells.

10. A method of treating a hematological condition, the method comprising administering to a human subject:
(a) the population of genetically engineered hematopoietic stem/progenitor cells or T cells of claim 7; and
(b) a therapeutically effective amount of at least one agent comprising an anti-CD38 antibody binding domain or an antibody or antibody fragment comprising the anti-CD38 binding domain.

11. The method of claim 10, wherein the at least one agent comprises a Chimeric Antigen Receptor-T (CAR-T) cell comprising the anti-CD38 antibody binding domain.

12. (canceled)

13. A chimeric antigen receptor (CAR) comprising a polypeptide comprising:
(a) one or more epitope binding fragments that binds to an epitope of one or more cell-surface lineage-specific proteins,
(b) a hinge domain,
(c) a transmembrane domain,
(d) a co-stimulatory domain, and
(e) a cytoplasmic signaling domain,
wherein one of the cell-surface lineage-specific proteins is CD38.

14. The CAR of claim 13, wherein the CAR comprises the amino acid sequence of any one of SEQ ID NO: 64 or 66, or a sequence that is at least 80%, at least 85%, at least 90%, at least 95%, at least 97%, at least 98%, or at least 99% identical to the amino acid sequence of SEQ ID NO: 64 or 66.

15. A cell expressing the CAR of claim 13.

16. (canceled)

17. (canceled)

18. A method of treating a hematological malignancy, the method comprising administering to a human subject:
(a) a population of genetically engineered hematopoietic stem/progenitor cells or T cells; and
(b) the cell of claim 15.

19. (canceled)

20. A pharmaceutical composition comprising the cells of any one of claim 15 and a pharmaceutically acceptable carrier.

21. A kit comprising the cell of claim 15, and optionally one or more cytotoxic agents targeting cell-surface antigens, the genes of which are edited in the hematopoietic stem/progenitor cells or T cells.

22. A polypeptide comprising an amino acid sequence that is at least 80% identical to the sequence set forth in SEQ ID NO: 52, wherein the polypeptide comprises a mutation at S274F and wherein the polypeptide has reduced binding to a therapeutic anti-CD38 antibody.

23. A nucleic acid encoding the polypeptide of claim 22.

24. A vector comprising the nucleic acid of claim 23.

25. A cell comprising the nucleic acid of claim 23.

26. A method of making a polypeptide, the method comprising culturing the cell of claim 25 under conditions that allow for the expression of the polypeptide and optionally isolating the polypeptide.

27. (canceled)

28. (canceled)
